(19) United States
(12) Patent Application Publication
(10) Pub. No.: US 2015/0359913 A1
(43) Pub. Date: Dec. 17, 2015

(54) TETRAZINES/TRANS-CYCLOOCTENES IN SOLID PHASE SYNTHESIS OF LABELED PEPTIDES

(71) Applicants: MEMORIAL SLOAN-KETTERING CANCER CENTER, New York, NY (US); THE GENERAL HOSPITAL CORPORATION, Boston, MA (US)

(72) Inventors: Thomas Reiner, Weehawken, NJ (US); Jason Stuart Lewis, New York, NY (US); Ralph Weissleder, West Peabody, MA (US); Brian Zeglis, New York, NY (US)

(21) Appl. No.: 14/763,380

(22) PCT Filed: Jan. 24, 2014
(86) PCT No.: PCT/US14/13023

§ 371 Date: Jul. 24, 2015

Related U.S. Application Data
(60) Provisional application No. 61/756,827, filed on Jan. 25, 2013.

(51) Int. Cl. A61K 51/10; A61K 51/04; A61K 49/04

(52) U.S. Cl. CPC A61K 51/10; A61K 49/04; A61K 51/044

(57) ABSTRACT

This invention is in the field of labeled peptide construction for medical treatment and analysis. The invention relates to synthetic labeled peptide compositions, methods of synthesis, and methods of use for the synthetic labeled peptide compositions for medical treatment, imaging, and research purposes.

### FIELD OF THE INVENTION

This invention is in the field of labeled peptide construction for medical treatment and analysis. The invention relates to synthetic labeled peptide compositions, methods of synthesis, and methods of use for the synthetic labeled peptide compositions for medical treatment, imaging, and research purposes. This invention relates to labeled diagnostic reagents and peptides, and methods for producing labeled diagnostic agents. Specifically, the invention relates to specific-binding peptides, methods and kits for making such peptides, and methods for using such peptides to image or treat sites in a mammalian body labeled with optical, fluorescent, or radioactive agents via a covalent label-binding moiety, which in some cases forms a complex with radioactive agents.

### BACKGROUND OF THE INVENTION

Translation of targeted peptide sequences to targeted radiolabeled or other labeled imaging agents can be time-consuming and often relies on serendipity rather than rational design. In certain cases, this is due to the limited number of chemical tools that are applicable as linkers to attach radiotracers or other labels to peptide sequences. The available reactions can require harsh conjugation conditions or sometimes do not meet chemoselectivity requirements. What is needed is an alternative to allow for consistent and controlled production of easily labeled and targeted peptides.

### SUMMARY OF THE INVENTION

This invention is in the field of labeled peptide construction for medical treatment and analysis. The invention relates to synthetic labeled peptide compositions, methods of synthesis, and methods of use for the synthetic labeled peptide compositions for medical treatment, imaging, and research purposes.

In one embodiment, the present invention contemplates a method of in vivo imaging, comprising a) ex vivo modification of an antibody with amine-reactive transcyclooctene to create a plurality of antibody-transcyclooctene conjugates; b) in vivo administration to a subject of the antibody-transcyclooctene conjugates wherein the subject comprises tissue reactive with the antibody; c) in vivo administration of a labeled tetrazine derivative, under conditions such that at least a portion of the administered labeled tetrazine derivative reacts with at least a portion of the plurality of conjugates to create an imaging reagent bound to the tissue reactive with the antibody; and d) imaging the tissue reactive with the antibody. In one embodiment, the subject is a human and the tissue reactive with the antibody is a tumor. In another embodiment, administration of conjugate at step b) is by intravenous injection into the blood of the subject. In another embodiment, sufficient time is provide prior to step c) during which a portion of the plurality of antibody conjugates binds to the tumor and at least a portion of unbound antibody conjugate clears from the blood. In yet another embodiment the labeled tetrazine derivative is labeled with a radiolabel. In yet another embodiment the antibody of step a) is a monoclonal antibody. In yet another embodiment the monoclonal antibody is a humanized antibody.

In one embodiment, the invention relates to a method of solid phase peptide synthesis, comprising a) synthesizing a peptide by stepwise addition of amino acids or analogs thereof while bound to a solid phase; and b) introducing a tetrazine amino acid at a specific site in said polypeptide during said solid phase peptide synthesis so as to create a tetrazine modified peptide. In one embodiment, said tetrazine amino acid is added after the first amino acid has been bound to a solid phase (and thus may be added to the next position or added at any position (second, third, forth, etc.) after the first amino acid has been bound). In one embodiment, said tetrazine amino acid is added before the last amino acid is added in the bound peptide. In one embodiment, said tetrazine amino acid is added as the last amino acid in the bound peptide. In one embodiment, the tetrazine-modified peptide comprises between 2 and 40 amino acids. In one embodiment, the method further comprises c) reacting said tetrazine modified peptide with a trans-cyclooctene containing labeled compound so as to create a labeled peptide. In one embodiment, said label of said labeled compound is a radiolabel. In one embodiment, said label of said labeled compound is a fluorescent label. In one embodiment, the invention relates to a library of at least six labeled peptides made as described above, wherein said label on each peptide is in a different position. In one embodiment, the invention relates to a library of at least three labeled peptides made as described above, wherein said label on each peptide is in a different position. In one embodiment, said tetrazine amino acid comprises an amino acid with an amine protecting group and a side chain with a linking group connected to a tetrazine moiety. In one embodiment, said amine protecting group is selected from the group consisting of Fmoc and Boc.

In one embodiment, the invention relates to a method of solid phase peptide synthesis, comprising a) synthesizing a peptide by stepwise addition of amino acids or analogs thereof while bound to a solid phase; and b) introducing a trans-cyclooctene amino acid at a specific site in said polypeptide during said solid phase peptide synthesis so as to create a trans-cyclooctene modified peptide. In one embodiment, said trans-cyclooctene amino acid is added after the first amino acid has been bound to a solid phase (and thus may be added to the next position or added at any position (second, third, forth, etc.) after the first amino acid has been bound). In one embodiment, said trans-cyclooctene amino acid is before the last amino acid is added in the bound peptide. In one embodiment, said trans-cyclooctene amino acid is added as the last amino acid in the bound peptide. In one embodiment, the trans-cyclooctene modified peptide comprises between 2 and 40 amino acids. In one embodiment, the method further comprises c) reacting said trans-cyclooctene amino acid containing peptide with a tetrazine containing labeled compound so as to create a labeled peptide. In one embodiment, said label of said labeled compound is a radiolabel. In one embodiment, said label of said labeled compound is a fluorescent label. In one embodiment, the invention relates to a library of at least six labeled peptides made as described above, wherein said label on each peptide is in a different position. In one embodiment, said trans-cyclooctene amino acid comprises an amino acid with an amine protecting group and a side chain with a linking group connected to a tetrazine moiety. In one embodiment, said amine protecting group is selected from the group consisting of Fmoc and Boc.

In one embodiment, the invention relates to a method for introducing a tetrazine amino acid at a specific site in a polypeptide during solid phase peptide synthesis, the method comprising: a) synthesizing a polypeptide by stepwise addition of amino acids or analogs thereof while bound to a solid phase until the position of the target tetrazine amino acid is to be added; b) deprotecting a first amino acid linked to the solid phase resin by removing protective chemical groups from the first acid; c) activating chemical groups on a tetrazine amino acid to prepare the tetrazine amino acid for coupling with the first amino acid; d) coupling the activated tetrazine amino acid to the deprotected first amino acid to form a peptide from the first and tetrazine amino acids; and e) successively deprotecting, activating, and coupling a plurality of amino acids or analogs thereof to said polypeptide until the desired tetrazine amino acid containing peptide is produced. In one embodiment, said method further comprises step f) successively combining said tetrazine amino acid containing peptide with a trans-cyclooctene containing labeled compound under such conditions that a tetrazine moiety of said tetrazine amino acid containing peptide reacts with said trans-cyclooctene moiety of said trans-cyclooctene containing labeled compound resulting in a labeled peptide. In one embodiment, said tetrazine containing labeled compound comprises ¹⁸F, ⁶⁴Cu, and ⁸⁹Zr. In one embodiment, said tetrazine amino acid comprises an amino acid with an N-terminal amine protecting group and a side chain with a linking group connected to a tetrazine moiety. In one embodiment, said N-terminal amine protecting group is selected from the group consisting of Fmoc and Boc. In one embodiment, said linking group is selected from the group consisting of short alkyl chains (2 or 4 CH₂-groups) or ethylene glycol units (3 or 6 glycol units).

In one embodiment, the invention relates to a method for introducing a trans-cyclooctene amino acid at a specific site in a polypeptide during solid phase peptide synthesis, the method comprising: a) synthesizing a polypeptide by stepwise addition of amino acids or analogs thereof while bound to a solid phase until the position of a target trans-cyclooctene acid is to be added; b) deprotecting a first amino acid linked to the solid phase resin by removing protective chemical groups from the first acid; c) activating chemical groups on a trans-cyclooctene acid to prepare the trans-cyclooctene acid for coupling with the first amino acid; d) coupling the activated trans-cyclooctene acid to the deprotected first amino acid to form a peptide from the first and trans-cyclooctene acids; and e) successively deprotecting, activating, and coupling a plurality of amino acids or analogs thereof to said polypeptide until the desired peptide is produced. In one embodiment, the method further comprises step f) successively combining said trans-cyclooctene amino acid containing peptide with a tetrazine containing labeled compound under such conditions that a trans-cyclooctene moiety of said trans-cyclooctene amino acid containing peptide reacts with said tetrazine moiety of said tetrazine containing labeled compound resulting in a labeled peptide. In one embodiment, said tetrazine containing labeled compound comprises ¹⁸F, ⁶⁴Cu, and ⁸⁹Zr. In one embodiment, the invention relates to a said trans-cyclooctene acid comprises an amino acid with an N-terminal amine protecting group and a side chain with a linking group connected to a tetrazine moiety. In one embodiment, the invention relates to a said N-terminal amine protecting group is selected from the group consisting of Fmoc and Boc. In one embodiment, the invention relates to a said linking group is selected from the group consisting of short alkyl chains (2 or 4 CH₂-groups) or ethylene glycol units (3 or 6 glycol units).

In one embodiment, the invention relates to a kit useful for preparing label containing peptides comprising: a) a trans-cyclooctene amino acid in a first compartment, wherein said trans-cyclooctene amino acid is capable of being integrated into a peptide during solid phase peptide synthesis, and b) a labeling agent comprising a tetrazine in a different compartment. In one embodiment, said label of said labeling agent is a radiolabel. In one embodiment, said label of said labeling agent is a fluorescent label. It is not intended that the present invention be limited to any particular type of compartment, however in one embodiment, said compartment can be a tube, vial, or any other type of carrier.

In one embodiment, the invention relates to a kit useful for preparing label containing peptides comprising: a) a tetrazine amino acid in a first compartment, wherein said tetrazine amino acid is capable of being integrated into a peptide during solid phase peptide synthesis, and b) a labeling agent comprising a trans-cyclooctene in a different compartment. In one embodiment, said label of said labeling agent is a radiolabel. In one embodiment, said label of said labeling agent is a fluorescent label. It is not intended that the present invention be limited to any particular type of compartment, however in one embodiment, said compartment can be a tube, vial, or any other type of carrier.

In one embodiment, the invention relates to a kit useful for preparing label containing peptide comprising: a) a trans-cyclooctene amino acid containing peptide in a first compartment, wherein said trans-cyclooctene amino acid has been integrated into said peptide during solid phase peptide synthesis, and b) a labeling agent comprising a tetrazine in a different compartment. In one embodiment, said label of said labeling agent is a radiolabel. In one embodiment, said label of said labeling agent is a fluorescent label. It is not intended that the present invention be limited to any particular type of compartment, however in one embodiment, said compartment can be a tube, vial, or any other type of carrier.

In one embodiment, the invention relates to a kit useful for preparing label containing peptides comprising: a) a tetrazine amino acid containing peptide in a first compartment, wherein said tetrazine amino acid has been integrated into said peptide during solid phase peptide synthesis, and b) a labeling agent comprising a trans-cyclooctene in a different compartment. In one embodiment, said label of said labeling agent is a radiolabel. In one embodiment, said label of said labeling agent is a fluorescent label. It is not intended that the present invention be limited to any particular type of compartment, however in one embodiment, said compartment can be a tube, vial, or any other type of carrier.

In one embodiment, the invention relates to a composition comprising a modified amino acid with the structure:

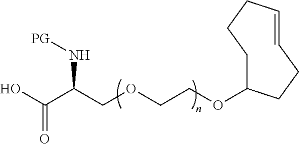

wherein n=3 or 6 and PG=a protecting group. In one embodiment, the invention relates to a composition comprising a modified amino acid with the structure:

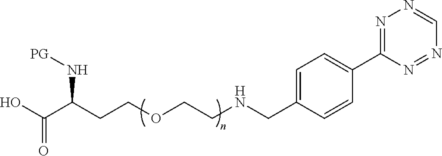

wherein n=3 or 6 and PG=a protecting group. In one embodiment, the invention relates to a composition comprising a modified amino acid with the structure:

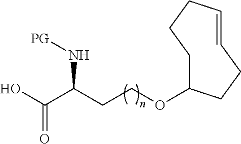

wherein n=2 or 4 and PG=a protecting group. In one embodiment, the invention relates to a composition comprising a modified amino acid with the structure:

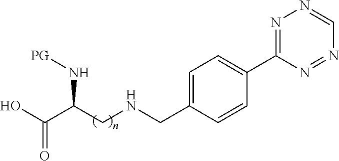

wherein n=2 or 4 and PG=a protecting group.

In one embodiment, the invention relates to a method of synthesizing a library of labeled peptides, the method comprising: a) providing a library of growing peptidic substrates attached to a solid support, each of which comprises a terminal primary alpha amine group; b) treating said library of peptidic substrates by addition of a modified amino acid, wherein the modified amino acid is selected from the group consisting of a tetrazine amino acid and trans-cyclooctene amino acid; c) successively deprotecting, activating, and coupling a plurality of amino acids or analogs thereof to said polypeptide until the desired modified amino acid containing peptide is produced; and d) successively combining said library of modified amino acid containing peptide with a label containing compound, under such conditions that a trans-cyclooctene moiety reacts with a tetrazine moiety resulting in a library labeled peptides, wherein said label containing compound has the alternative reactive group of trans-cyclooctene or tetrazine compared to said modified amino acid. In one embodiment, the addition of a modified amino acid comprises the replacement of at least one natural amino acid in the sequence of the target peptide. In one embodiment, the addition of a modified amino acid comprises the addition of at least one modified amino acid into the sequence of target peptide. It is not intended that the present invention be limited to any particular size of library. However, in one embodiment, said library comprises at least three peptides wherein label is in a different position in each peptide sequence. In one embodiment, said library comprises at least five peptides wherein label is in a different position in each peptide sequence. In one embodiment, said library comprises at least 10 peptides wherein label is in a different position in each peptide sequence. In one embodiment, said library comprises at least 50 peptides wherein label is in a different position in each peptide sequence. In one embodiment, said library comprises at least 100 peptides wherein label is in a different position in each peptide sequence. In one embodiment, said library comprises at least 500 peptides wherein label is in a different position in each peptide sequence. In one embodiment, said library comprises at least 1000 peptides wherein label is in a different position in each peptide sequence. In one embodiment, said library comprises more than 1000 peptides wherein label is in a different position in each peptide sequence. In one embodiment, said library of target compounds comprises at least 1,000 different target compounds.

It is not intended that present invention be limited to the modification of specific amino acids to trans-cyclooctene or tetrazine containing amino acids, but considers modification to all known amino acids. It is not intended that trans-cyclooctene or tetrazine modification be limited to natural amino acids. It is not intended that present invention be limited to the replacement with a matching trans-cyclooctene or tetrazine containing amino acid, for example where a peptide has a lysine, said lysine may be replaced by a cyclooctene or tetrazine containing lysine or some other cyclooctene or tetrazine containing amino acid. In one embodiment of the present invention, the creation of a library of peptides comprises a strictly faithful substitution pattern wherein an amino acid is replaced with the same amino acid containing a cyclooctene or tetrazine moiety. In one embodiment of the present invention, the creation of a library of peptides comprises a conservative substitution pattern wherein an amino acid is replaced with the same or similar amino acid containing a cyclooctene or tetrazine moiety. In one embodiment, said conservative substitution pattern comprises substitution of modified amino acids from with each of the groups: aromatic (Phe, Tyr, Trp), large aliphatics with methionine and cystine (Val, Ile, Leu, Met, Cys), small amino-acids (Gly, Ser, Thr, Asp, Asn, Ala, Glu, Gln, Pro) and ionizable basic amino acids (His, Arg, Lys). For example, if the original template peptide had a leucine at the fifth position, then an acceptable conservative substitution would be to incorporate a cyclooctene or tetrazine containing isoleucine at the fifth position. In one embodiment of the present invention, the creation of a library of peptides comprises a strictly faithful substitution pattern wherein an amino acid is replaced with the same, similar or different amino acid containing a cyclooctene or tetrazine moiety. In one embodiment of the present invention, the creation of a library of peptides comprises the addition of an extra amino acid containing a cyclooctene or tetrazine moiety to the peptide sequence. For example, if the original peptide sequence was GTLIFGWY (SEQ ID No:1), then an addition of an extra amino acid containing a cyclooctene (o) or tetrazine (τ) moiety to the peptide sequence would result in a synthesized peptide with a sequence, for example GTLIFQ^{o}GWY (SEQ ID No:2) or GTLIFQ^{τ}GWY (SEQ ID No:3), both where the extra amino acid, a glutamine containing a cyclooctene (o) or tetrazine (τ) moiety in this case, were added between phenylalanine and glycine. This is in contrast with the substitution paradigm wherein a sequence GTLIFGWY (SEQ ID No: 1), would have a replacement at some position, such as GTLIFQ^{o}WY (SEQ ID No: 4) or GTLIFQ^{τ}WY (SEQ ID No: 5), wherein the glycine from the original sequence. In some embodiments of the invention, the amino acid containing a cyclooctene moiety is represented simply by the letter omicron (o) and amino acid containing a tetrazine moiety is represented simply by the letter tau (τ) in the peptide sequence, for example as shown in FIG. 2F, with the sequence GAτFV (SEQ ID No: 6).

In one embodiment, the invention relates to a method of carrying out peptide synthesis comprising: deprotecting an Fmoc-protected amino acid with piperazine and hydroxybenzotriazole (HOBt) while applying microwave irradiation to the deprotection reaction.

In one embodiment, the invention relates to a method of carrying out peptide synthesis comprising: deprotecting a Boc-protected amino acid with trifluoroacetic acid or mixtures of trifluoroacetic acid and dichloromethane.

In one embodiment, the invention relates to a method of carrying out peptide synthesis comprising: deprotecting a Boc-protected amino acid under simultaneous cleavage of the polypeptide from the resin with trifluoroacetic acid containing small quantities of water (between 0 and 10%) while applying microwave irradiation to the deprotection/cleavage reaction.

In one embodiment, the invention relates to a method of carrying out peptide synthesis comprising: cleavage of the polypeptide from the resin with trifluoroacetic acid containing small quantities of water (between 0 and 10%) while applying microwave irradiation to the cleavage reaction.

In one aspect, the present invention provides a pharmaceutical composition comprising the imaging agent as described above, together with a biocompatible carrier, in a form suitable for mammalian administration. In a preferred embodiment, the pharmaceutical composition is a radiopharmaceutical composition.

The imaging agents of the invention are useful for in vivo imaging. Accordingly, in another aspect, the present invention provides an imaging agent of the invention for use in an in vivo diagnostic or imaging method, e.g. SPECT or PET. This aspect of the invention also provides a method for the in vivo diagnosis or imaging in a subject with a condition in need of diagnosis or imaging. Said subject is preferably a mammal and most preferably a human. In an alternative embodiment, this aspect of the invention furthermore provides for the use of the imaging agent of the invention for imaging in vivo of a condition in a subject wherein said subject is previously administered with the pharmaceutical composition of the invention. By “previously administered” is meant that the step involving the clinician, wherein the pharmaceutical is given to the patient e.g., intravenous injection, has already been carried out. This aspect of the invention also encompasses use of the imaging agent of the previous embodiment for the manufacture of pharmaceutical for the diagnostic imaging. In another aspect, the invention provides a method of monitoring the effect of treatment of a human or animal body with a drug, said method comprising administering to said body an imaging agent of the invention and detecting the uptake of said imaging agent, said administration and detection optionally but preferably being effected repeatedly, e.g. before, during and after treatment with said drug.

In one embodiment, the invention relates to designing peptides, which bind to cell surfaces without being internalized. They will then be injected with a complementary tracer (which can be anything from the list below) and clicked in vivo. This is particularly useful if toxic compounds are being delivered, which can cause serious side effects if they circulate longer.

Other than radiolabels, other imaging labels or tracers paired with peptides could include:

a) Fluorophores (e.g. Cy5, Cy5.5, Cy7, Alexa Fluor dyes, BODIPY dyes, Coumarin dyes, Fluorescin dyes)

b) Chromophores, including auxochromes and halochromes (e.g. beta-carotin, phenolphthalein, crystal violet, Orange G, Victoria Blue, Congo Red)

c) FRET pairs (e.g. Cy2-Cy3, Alexa Fluor 647-Alexa Fluor 750, CFP-YFP, GFP-mRFP, FITC-TRITC)

d) MRI imaging agents (e.g. Omniscan, Gd³⁺ binding chelators, Iron Oxide chelators, small molecules or nanoparticles)

e) CT contrast agents (e.g. Iohexol, Iopromide, Diatrizoate)

f) SPECT imaging agents (e.g. ¹²³I-MIBG, ¹³¹I-MIBG, ^{99m}Tc-HMPAO, ^{99m}Tc-tetrofosmin)

g) Cytotoxic materials (including alkylating agents and anti-metabolites, plant alkaloids and terpenoids, either Organometallic or organic or inorganic). (e.g. cis-Pt, carbo-Pt, taxol, cyclophosphamide, decetaxel)

h) Cherenkov-active isotopes (e.g. ¹⁸F, ⁶⁴Cu, ⁶⁸Ga)

i) Phosphorescent probes (e.g. containing ZnS, CaS, SrAl₂O₄, Ln silicates)

j) Luminescent probes (e.g. chemiluminescence, bioluminescence, electochemiluminescence, electroluminescence, crystallomuminescence, electrochemiluminescence, photoluminescence, radioluminescence, sonoluminescence, thermoluminescence)

k) Probes that represent a key component for the generation of Luminescence (e.g. luciferin, luciferase, ATP)

l) Photodynamic therapeutics and diagnostics (e.g. photosensitizers, photocleavable groups)

m) Nanoparticles (e.g. dextran-based nanoparticles, crosslinked iron oxide nanoparticles, silica-based nanoparticles)

n) Quantum dots (e.g. Cadmium and Cadmium-Free Quantum dots, 1-1000 nm in size)

o) Redox-active organic or organometallic complexes (e.g. Ferrocene, Ferrocenium, cobaltocene, cobaltocenium, other metals or transition metal complexes)

p) Heat-sensitive materials (e.g. fluorite, feldspars, quartz)

q) Cold-sensitive materials (e.g. Bis(diethylammonium)tetrachlorocuprate, Leuco dye, spirolactones, fluorans, spiropyrans)

r) Sensors for Reactive Oxygen species (ROS) (e.g. trans-1-(2′-Methoxyvinyl)pyrene, 2-hydroxy-5-(triphenylphosphonium)hexylethidium, Amplex Red, 3′-(p-hydroxyphenyl) fluorescein)

s) Enzymes, peptides or other biomolecules (fluorescent or biologically active) (e.g. GFP, RFP, Cytochrome P50, HSP90, Somatostatin, Neurotensin Y, Substance P)

t) Small molecules markers (e.g. AZD2281, Taxol, Aspirin, ADP, ATP, NADP, Staurosporine, Estrogen)

u) Lanthanides (e.g. Lanthan, Cerium, Praesodymium, Neudymium, Promethium, Samarium, Europium, Gadolinium, Terbium, Dysprosium, Holmium, Erbium, Thumium, Ytterbium, Lutetium)

v) Actinides (e.g. Actinium, Thorium, Protactinium, Neptunium, Einsteinium)

w) Isotopically enriched non-radioactive isotopes (lanthanides, transition metals, metals). (e.g. ¹⁴²Nd, ¹⁴⁸Nd, ¹⁶⁶Er, ¹⁶⁸Yb, ¹⁷⁶Yb, ¹³⁹La).

### DEFINITIONS

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “patient” or “subject” refers to any living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

As used herein, the term “trans-cyclooctene” refers to at least an eight membered ring with a “trans” double bone. One example of such a compound is show with the structure:

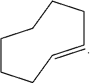

As used herein, the term “tetrazine” refers to at least a six-membered aromatic ring containing four nitrogen atoms with the molecular formula C₂H₂N₄. The name tetrazine is used in the nomenclature of derivatives of this compound. Three core-ring isomers exist: 1,2,3,4-tetrazines, 1,2,3,5-tetrazines and 1,2,4,5-tetrazines. One example of such a compound is show with the structures:

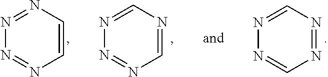

As used herein, the term “Diels-Alder reaction” refers to an organic chemical reaction (specifically, a cycloaddition) between a conjugated diene and a substituted alkene, commonly termed the dienophile, to form a substituted cyclohexene system. One example of a Diels-Alder reaction pair is tetrazine and a trans-cyclooctene wherein a diene is tetrazine and trans-cyclooctene is the dienophile.

As used herein, the term “modified amino acid” refers to an amino acid that contains either tetrazine or trans-cyclooctene. In one example, such a modified amino acid has one following structures:

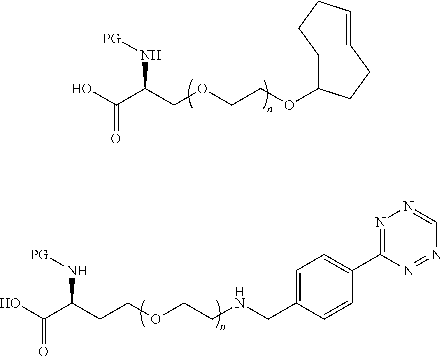

wherein n=3 or 6 and PG=a protecting group, or

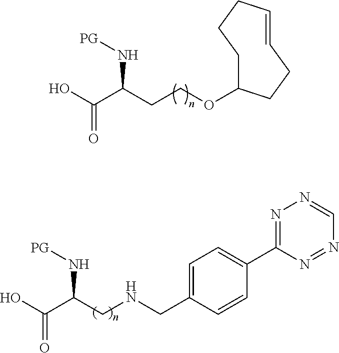

wherein n=2 or 4 and PG=a protecting group.

As used herein, the term “short alkyl chains” refers to either 2 to 4 CH₂ groups.

As used herein, the term “ethylene glycol units” refers to units with the structure of —OCH₂CH₂—.

As used herein, the term “protecting group” refers to a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Protecting groups are well known to those skilled in the art and are suitably chosen from, for amine groups: Boc (where Boc is tert-butyloxycarbonyl), Fmoc (where Fmoc is fluorenylmethoxycarbonyl or 9-fluorenylmethyloxycarbonyl), trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl); and for carboxyl groups: methyl ester, tert-butyl ester or benzyl ester. For hydroxyl groups, suitable protecting groups are methyl, ethyl or tert-butyl; alkoxymethyl or alkoxyethyl; benzyl; acetyl; benzoyl; trityl (Trt) or trialkylsilyl such as tetrabutyldimethylsilyl. For thiol groups, suitable protecting groups are trityl and 4-methoxybenzyl. The use of further protecting groups are described in ‘Protective Groups in Organic Synthesis’, Theorodora W. Greene and Peter G. M. Wuts, (Third Edition, John Wiley & Sons, 1999) [1].

As used herein, the term “biocompatible carrier” refers to a fluid, especially a liquid, in which the imaging agent is suspended or dissolved, such that the composition is physiologically tolerable, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier medium may also comprise biocompatible organic solvents such as ethanol. Such organic solvents are useful to solubilise more lipophilic compounds or formulations. Preferably, the biocompatible carrier medium is pyrogen-free water for injection, isotonic saline or an aqueous ethanol solution. The pH of the biocompatible carrier medium for intravenous injection is suitably in the range 4.0 to 10.5.

As used herein, the term “solid-phase synthesis” or “SPPS” refers to chemical synthesis of peptides. SPPS allows the synthesis of natural peptides that are difficult to express in bacteria, the incorporation of unnatural amino acids, peptide/protein backbone modification, and the synthesis of D-proteins, which consist of D-amino acids. Small solid beads, insoluble yet porous, are treated with functional units (‘linkers’) on which peptide chains can be built. The peptide will remain covalently attached to the bead until cleaved from it by a reagent such as anhydrous hydrogen fluoride or trifluoroacetic acid. The peptide is thus ‘immobilized’ on the solid-phase and can be retained during a filtration process, whereas liquid-phase reagents and by-products of synthesis are flushed away. The general principle of SPPS is one of repeated cycles of coupling-wash-deprotection-wash. The free N-terminal amine of a solid-phase attached peptide is coupled to a single N-protected amino acid unit. This unit is then deprotected, revealing a new N-terminal amine to which a further amino acid may be attached. The superiority of this technique partially lies in the ability to perform wash cycles after each reaction, removing excess reagent with all of the growing peptide of interest remaining covalently attached to the insoluble resin. The overwhelmingly important consideration is to generate extremely high yield in each step. For example, if each coupling step were to have 99% yield, a 26-amino acid peptide would be synthesized in 77% final yield (assuming 100% yield in each deprotection); if each step were 95%, it would be synthesized in 25% yield. Thus, each amino acid is added in major excess (2-10×) and coupling amino acids together is highly optimized by a series of well-characterized agents. There are two majorly used forms of SPPS—Fmoc and Boc. Unlike ribosome protein synthesis, solid-phase peptide synthesis proceeds in a C-terminal to N-terminal fashion. The N-termini of amino acid monomers is protected by either of these two groups and added onto a deprotected amino acid chain. Automated synthesizers are available for both techniques, though many research groups continue to perform SPPS manually. SPPS is limited by yields, and typically peptides and proteins in the range of 70 amino acids are pushing the limits of synthetic accessibility. Synthetic difficulty also is sequence dependent; typically amyloid peptides and proteins are difficult to make. Longer lengths can be accessed by using native chemical ligation to couple two peptides together with quantitative yields.

As used herein, the term “chelator” refers to a chemical compound in the form of a heterocyclic ring or surrounding structure containing a metal ion attached by coordinate bonds to at least two nonmetal ions.

As used herein, the term “derivative” refers to any chemically or biologically modified version of a chemical compound that is structurally similar to a parent compound and (actually or theoretically) derivable from that parent compound. A “derivative” differs from an “analogue” in that a parent compound may be the starting material to generate a “derivative,” whereas the parent compound may not necessarily be used as the starting material to generate an “analogue.” An analogue may have different chemical or physical properties of the parent compound. For example, the derivative may be more hydrophilic or it may have altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The term “derivative” also includes conjugates, and prodrugs of a parent compound (i.e., chemically modified derivatives that can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher et al., Advanced Drug Delivery Reviews 19 (1996) 115 [2] incorporated herein by reference. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, for example carboxylic acid groups, can form, for example, alkali metal salts or alkaline earth metal salts (e.g., sodium salts, potassium salts, magnesium salts and calcium salts, and also salts with physiologically tolerable quaternary ammonium ions and acid addition salts with ammonia and physiologically tolerable organic amines such as, for example, triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. Compounds that simultaneously contain a basic group and an acidic group, for example a carboxyl group in addition to basic nitrogen atoms, can be present as zwitterions. Salts can be obtained by customary methods known to those skilled in the art, for example by combining a compound with an inorganic or organic acid or base in a solvent or diluent, or from other salts by cation exchange or anion exchange

### DESCRIPTION OF THE FIGURES

The accompanying figures, which are incorporated into and form a part of the specification, illustrate several embodiments of the present invention and, together with the description, serve to explain the principles of the invention. The figures are only for the purpose of illustrating a preferred embodiment of the invention and are not to be construed as limiting the invention.

FIG. 1 show a conceptual design of tetrazine-amino acid incorporation into peptides. (A) Late stage orthogonalization, a common synthetic route for targeted peptides: Peptides are synthesized using solid phase peptide synthesis and most commonly derivatized using functional groups selective or semi-selective for biogenic amino acids. (B) Early stage orthogonalization of peptides via Tetrazine amino acids: Tetrazine amino acids will be used identically to biogenic amino acids and incorporated during solid phase peptide. After cleavage of the peptide from the solid support, tetrazine amino acids can serve as chemically orthogonal linkers and react in radiochemically relevant concentrations, yielding targeted imaging agents fast and selectively.

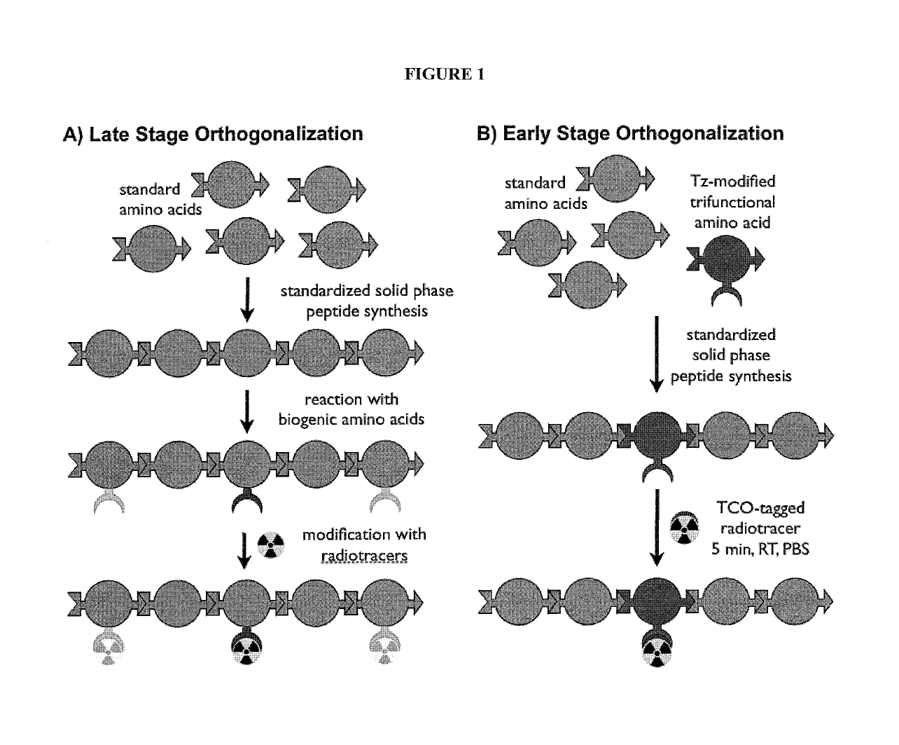

FIG. 2 demonstrates the Stability of tetrazine amino acids under solid phase peptide synthesis conditions. (A) Stability of tetrazine under N-Boc deprotection conditions; (B) Stability of tetrazine amino acids under amino acid coupling conditions; (C) Stability of tetrazine amino acids under resin cleavage conditions; (E) Structure of the novel tetrazine-pentapeptide GAτFV (SEQ ID No:6); (F) HPLC-trace of the pentapeptide GAτFV (SEQ ID No:6) before and after HPLC-purification.

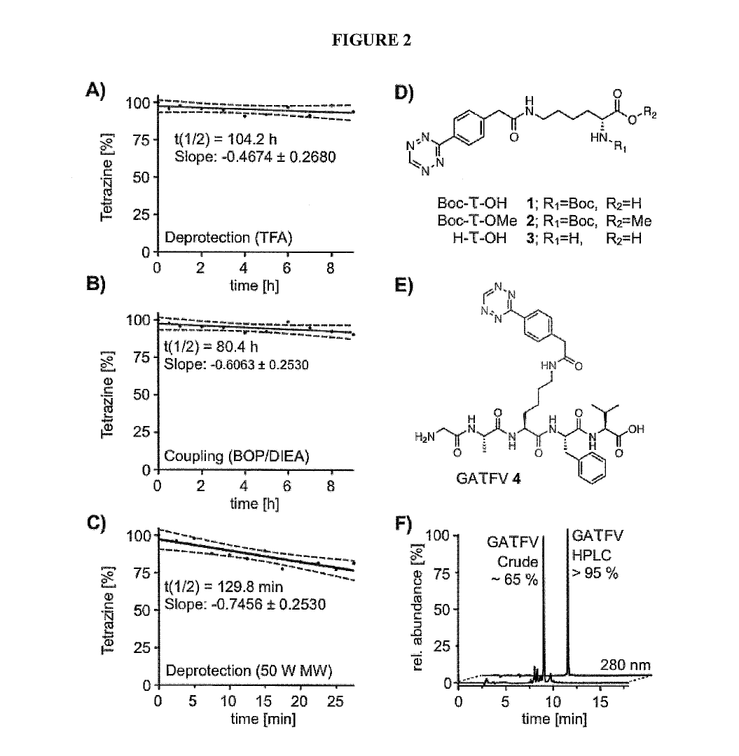

FIG. 3 shows an illustrated sequence of tetrazine-peptide synthesis on solid phase. Deprotection conditions: TFA/DCM=1/1, 1×5 and 1×20 min. Coupling conditions: BOP/DIPEA, 60 min. Resin Cleavage Conditions: TFA/H₂O=97.5/2.5, 50 W MW, 2×5 min. (A) Stability of tetrazine under N-Boc deprotection conditions; (B) Stability of tetrazine amino acids under amino acid coupling conditions; (C) Stability of tetrazine amino acids under resin cleavage conditions; (E) Structure of the novel tetrazine-pentapeptide GAτFV (SEQ ID No:6); (F) HPLC-trace of the pentapeptide GAτFV (SEQ ID No:6) before and after HPLC-purification.

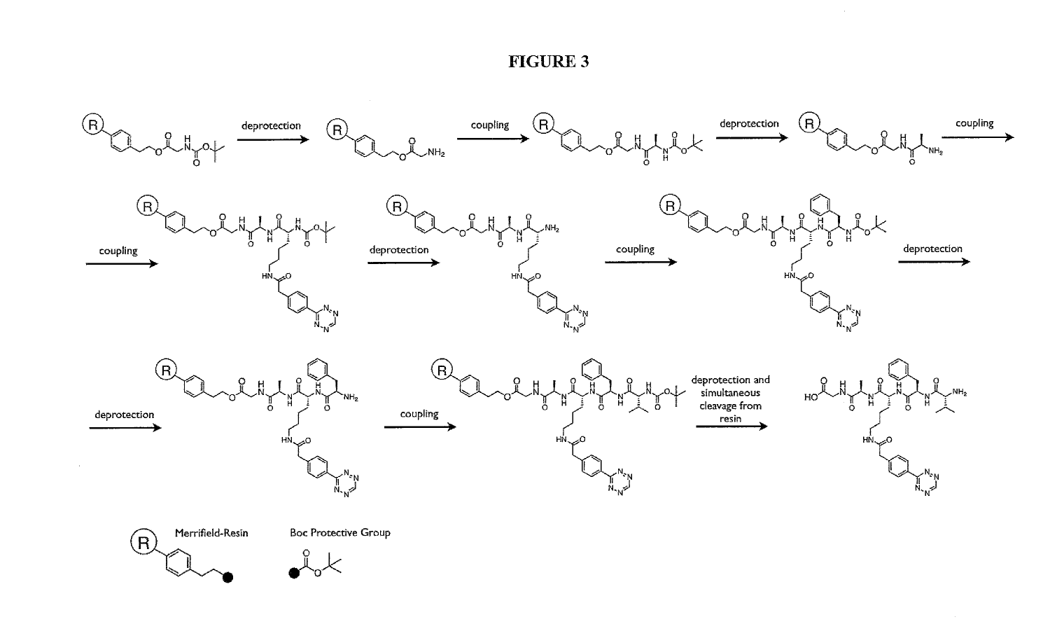

FIG. 4 shows preliminary results with site-selective backbone modification of peptide-reporters. (A) Schematic synthesis of radiolabeled probes using rapid and selective tetrazine/trans-cyclooctene chemistry. (B) Synthetic scheme for the synthesis of an ¹⁸F-radiolabeled Exendin-4 analog using tetrazine and trans-cyclooctene chemistry. (C) PET-CT and (D) PET only scans of an ¹⁸F-labeled Exendin-4 analog in a C57BL/6 mouse bearing 916-1 tumor xenografts. 916-1=tumor xenograft; I=intestines; K=kidney; B=bladder.

FIG. 5 shows GFP and NIR-probe colocalization in MIP-GFP mice. (A) Green, MIP-GFP; (B) Red, side chain modified NIR-probe (position K12); (C) Green, MIP-GFP; (D) Red, N-terminal modified NIR-probe. The arrows point to single islets.

FIG. 6 shows the development and use of new tetrazines/trans-cyclooctene amino acids for peptide radiolabeling. (A) Proposed Fmoc- and Boc-protected trans-cyclooctene and tetrazine amino acids. (B) Schematic representation of the transferrin-receptor binding peptide CTRIGPSVC (SEQ ID NO: 7). (C) Sequence, length and molecular weight of a transferrin-receptor binding peptide.

FIG. 7 shows the somatostatin-14. (A) Schematic representation of the secondary structure of somatostatin-14. (B) Sequence (SEQ ID NO: 8), length and molecular weight of somatostatin-14.

FIG. 8 shows novel and literature-known tetrazine/trans-cyclooctene based PET radioligands. (A) Examples of novel Zr-chelating trans-cyclooctene and (B) Zr-chelating tetrazine linkers, including their respective HPLC/MS traces. (C) Literature-known and in-house prepared and trans-cyclooctene and tetrazine PET linkers. ^{a}Li et al., 2010, [3]; ^{b}Keliher et al., 2011, [4]; ^{c}Zeglis et al., 2011, [5]; ^{d}unpublished results.

FIG. 9 shows a synthesis of NOTA-TCO. i) 1M NaHCO₃, 5 eq amine; ii) TFA, 100%, 1 h, evaporation; iii) TCO-NHS, Et₃N, DMSO. Bottom: HPLC and MS traces of NOTA-TCO

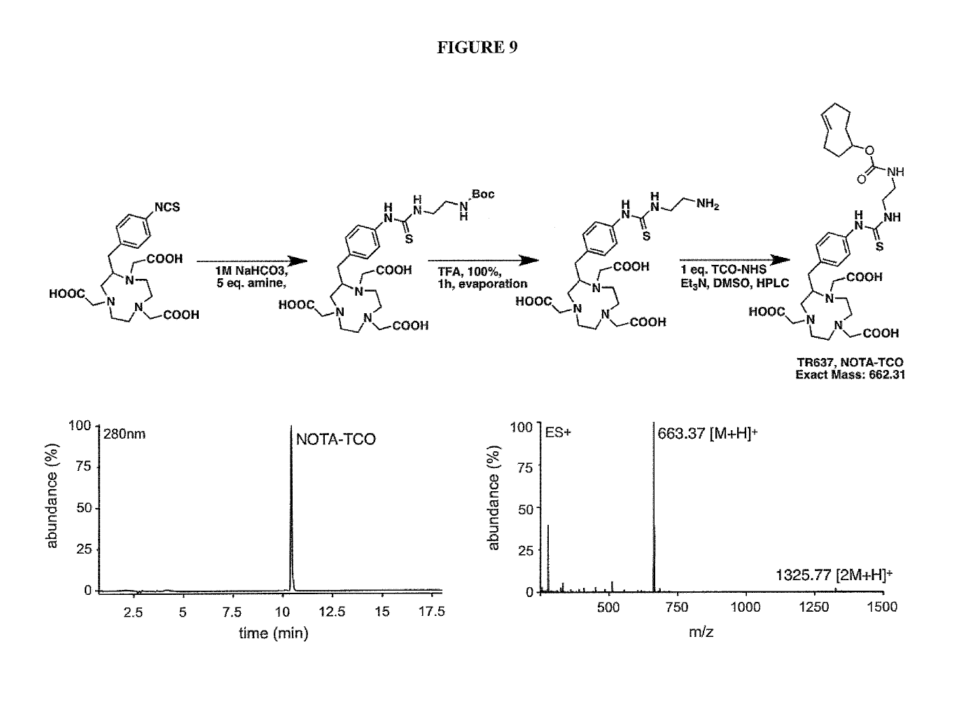

FIG. 10 shows a Synthesis of ⁸⁹Zr-TCO, a trans-cyclooctene labeled PET tracer. i) 5 eq. Et₃N, H₂O/AcN=1/1, 90 min, RT; ii) PBS, RT, 30 min.

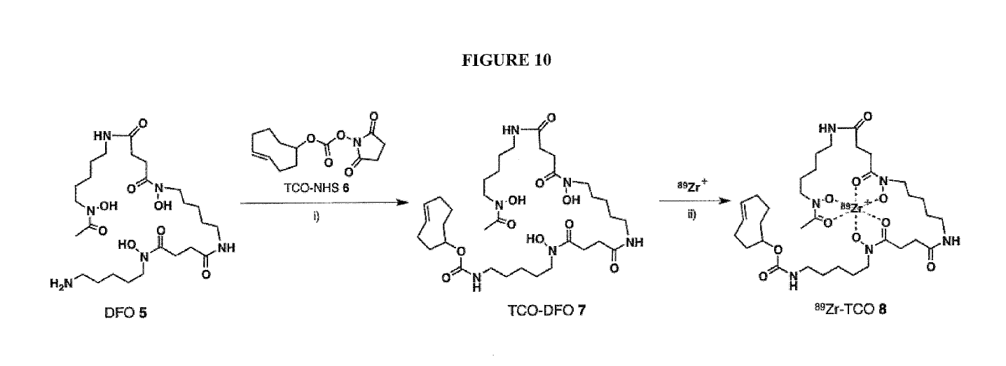

FIG. 11 shows a multiplexed synthesis of targeted tetrazine/trans-cyclooctene peptides and peptidomimetics. (A) Parallelized synthesis of tetrazine or trans-cyclooctene peptides. A magnetic scavenger resin will be used to remove excess cold unlabeled material before filtration of the radiolabels. (B) Staggered, parallel HPLC will determine radiochemical yield, purity and solubility of the radiolabeled peptides before binding assays will be performed. (C) Lead candidates will be tested in animal models to determine their metabolic stability, biodistribution and blood half-life. CHI-index=chromatographic hydrophobicity index.

FIG. 12 depicts the inverse electron-demand Diels-Alder [4+2] cycloaddition click ligation (IEDDA) between tetrazine and transcyclooctene.

FIG. 13 depicts the click chemistry-based pre-targeting system. (A) IEDDA click reaction between A33-TCO and [⁶⁴Cu]-Tz-Bn-NOTA. (B) Illustration of the 4 step pre-targeting methodology.

FIG. 14 depicts the results of a MSKCC clinical trial imaging patients with colorectal cancer with ¹²⁴I-huA33. (A) quantitative plasma, tumor and colon clearance curves obtained after injection of 10 mi ¹²⁴I-huA33 into a patient with colon cancer, metastatic to the liver (red arrows indicate potential ⁶⁴Cu-Tz-NOTA injection time points). (B) graph of tumor-to-plasma and tumor-to-colon activity ratios over the course of the imaging experiment. (C) PET image of a patient obtained at 5 days after injection with ¹²⁴I-huA33, showing uptake in tumors in liver and splenic flexure (solid black arrows) along with background intestinal uptake (dashed black arrows).

FIG. 15 depicts the synthesis of [⁶⁴Cu]-Tz-Bn-NOTA.

FIG. 16 depicts PET images of the [⁶⁴Cu]-Tz-Bn-NOTA/A33-TCO pre-targeting strategy. Mice bearing subcutaneous SW1222 xenografts (100-150 mm³, arrow) were administered A33-TCO (10 μg) via tail vein injection. After 24 hours the same mice were administered [⁶⁴Cu]-Tz-Bn-NOTA (10.2-12.0 MBq [275-325 μCi], 1.2-1.4 μg, for 2.5-2.8 Tz:A33 ratio) via tail vein injection. Transverse (top) and coronal (bottom) planar images intersect the center of the tumors.

FIG. 17 depicts autoradiography, histology and fluorescence microscopy of resected SW1222 xenografts from the multimodality pre-targeting experiment. (A) fluorescence microscopy indicating A33-TCO-AF680 localization (red); (B) autoradiography indicating ⁶⁴Cu-Tz-Bn-NOTA; (C) hematoxylin and eosin staining; and (D) fluorescence microscopy images with A33-TCO-AF680 (red) and 4′,6-diamidino-2-phenylindolel (DAPI) nuclear counterstain (blue) corresponding to the box in (C).

FIG. 18 depicts PET imaging with (A)⁶⁴Cu-NOTA-huA33 and (B)⁸⁹Zr-DFO-huA33. Mice bearing subcutaneous SW122 xenografts (100-150 mm³, white arrow) were administered [⁶⁴Cu]-NOTA-huA33 and (B) [⁸⁹Zr]-DFO-huA33 (275-325 μCi) via tail vein injection and imaged between 4 and 120 hours post-injection. The transverse and coronal planar images intersect the center of the tumors.

Table 1 provides biodistribution data for in vivo ⁶⁴Cu pre-targeting experiments. Values are represented as % ID/g±SD. Mice (n=4) bearing subcutaneous SW1222 xenografts were administered huA33-TCO (100 μg) via tail vein injection. After 24 hours the same mice were administered ⁶⁴Cu-Tz-Bn-NOTA (0.55-0.75 MBq [15-20 μCi] via tail vein injection (t=0).

Table 2 provides dosimetry calculations for the ⁶⁴Cu pre-targeting, ⁶⁴Cu-NOTA-huA33 and ⁸⁹Zr-DFO-huA33 imaging constructs. Mean organ absorbed doses and effective dose are expressed in mGy/MBq and mSv/MBq, respectively. 100 μg huA33-TCO was injected 24 hours prior to injection of ⁶⁴Cu-Tz-NOTA. 5 μg ⁶⁴Cu-NOTA-huA33 was administered per injection. 5 μg ⁸⁹Zr-DFO-huA33 was administered per injection.

### DETAILED DESCRIPTION OF THE INVENTION

The generic invention involves preparing a labeled amino acid monomer that can be integrated during normal solid phase peptide synthesis into a known part of the peptide. This allows for rational design of a library that one can screen. One specific invention involves particular peptides, e.g. transferrin receptor imaging agent for glioblastoma and somatostatin, a peptide hormone.

### Peptide Synthesis

The present invention relates to solid-phase peptide synthesis (SPPS), and in particular relates to microwave-assisted techniques for SPPS. Peptide synthesis may include the methods described in U.S. Pat. Nos. 7,393,920 [6] and 8,314,208 [7], hereby incorporated by reference.

The early part of the twentieth century saw the birth of a novel concept in scientific research in that synthetically produced peptides could greatly facilitate the study of the relationship between chemical structure and biological activity. Until that time, the study of structure-activity relationships between peptides and their biological function had been carried out using purified, naturally occurring peptides. Such early, solution-based techniques for peptide purification were plagued with problems, however, such as low product yield, contamination with impurities, their labor-intensive nature and the unpredictable solubility characteristics of some peptides. During the first half of the twentieth century, some solution-based synthesis techniques were able to produce certain “difficult” peptides, but only by pushing known techniques to their limits. The increasing demand for higher peptide yield and purity resulted in a breakthrough technique first presented in 1963 for synthesizing peptides directly from amino acids, now referred to as solid-phase peptide synthesis (SPPS).

The drawbacks inherent in solution-based peptide synthesis have resulted in the near-exclusive use of SPPS for peptide synthesis. Solid phase coupling offers a greater ease of reagent separation, eliminates the loss of product due to conventional chemistries (evaporation, recrystalization, etc.), and allows for the forced completion of the reactions by adding excess reagents.

Peptides are defined as small proteins of two or more amino acids linked by the carboxyl group of one to the amino group of another. Accordingly, at its basic level, peptide synthesis of whatever type comprises the repeated steps of adding amino acid molecules to one another or to an existing peptide chain of acids.

The synthetic production of peptides is an immeasurably valuable tool in the field of scientific research for many reasons. For example, some antiviral vaccines that exist for influenza and the human immunodeficiency virus (HIV) are peptide-based. Likewise, some work has been done with antibacterial peptide-based vaccines (diphtheria and cholera toxins). Synthetically altered peptides can be labeled with tracers, such as radioactive isotopes, and used to elucidate the quantity, location, and mechanism of action of the native peptide's biological acceptor (known as a receptor). This information can then be used to design better drugs that act through that receptor. Peptides can also be used for antigenic purposes, such as peptide-based antibodies to identify the protein of a newly discovered gene. Finally, some peptides may be causative agents of disease. For example, an error in the biological processing of the beta-amyloid protein leads to the “tangling” of neuron fibers in the brain, forming neuritic plaques. The presence of these plaques is a pathologic hallmark of Alzheimer's Disease. Synthetic production of the precursor, or parent molecule, of beta-amyloid facilitates the study of Alzheimer's Disease.

These are, of course, only a few of the wide variety of topics and investigative bases that make peptide synthesis a fundamental scientific tool.

The basic principle for SPPS is the stepwise addition of amino acids to a growing polypeptide chain that is anchored via a linker molecule to a solid phase particle, which allows for cleavage and purification once the coupling phase is complete. Briefly, a solid phase resin support and a starting amino acid are attached to one another via a linker molecule. Such resin-linker-acid matrices are commercially available (e.g., Calbiochem, a brand of EMD Biosciences, an affiliate of Merck KGaA of Darmstadt, Germany; or ORPEGEN Pharma of Heidelberg, Germany, for example). The starting amino acid is protected by a chemical group at its amino terminus, and may also have a chemical side-chain protecting group. The protecting groups prevent undesired or deleterious reactions from taking place at the alpha-amino group during the formation of a new peptide bond between the unprotected carboxyl group of the free amino acid and the deprotected alpha-amino of the growing peptide chain. A series of chemical steps subsequently deprotect the amino acid and prepare the next amino acid in the chain for coupling to the last. Stated differently, “protecting” an acid prevents undesired side or competing reactions, and “deprotecting” an acid makes its functional group(s) available for the desired reaction.

When the desired sequence of amino acids is achieved, the peptide is cleaved from the solid phase support at the linker molecule. This technique consists of many repetitive steps making automation attractive whenever possible.

Many choices exist for the various steps of SPPS, beginning with the type of reaction. SPPS may be carried out using a continuous flow method or a batch flow method. Continuous flow is useful because it permits real-time monitoring of reaction progress via a spectrophotometer. However, continuous flow has two distinct disadvantages in that the reagents in contact with the peptide on the resin are diluted, and scale is more limited due to physical size constraints of the solid phase resin. Batch flow occurs in a filter reaction vessel and is useful because reactants are accessible and can be added manually or automatically.

Other choices exist for chemically protecting the alpha-amino terminus. A first is known as “Boc” (tert-butoxycarbonyl). Although reagents for the Boc method are relatively inexpensive, they are highly corrosive and require expensive equipment. The preferred alternative is the “Fmoc” (9-fluorenylmethyloxycarbonyl) protection scheme, which uses less corrosive, although more expensive, reagents.

For SPPS, solid support phases are usually polystyrene suspensions; more recently, polymer supports such as polyamide have also been used. Preparation of the solid phase support includes “solvating” it in an appropriate solvent (dimethyl formamide, or DMF, for example). The solid phase support tends to swell considerably in volume during solvation, which increases the surface area available to carry out peptide synthesis. As mentioned previously, a linker molecule connects the amino acid chain to the solid phase resin. Linker molecules are designed such that eventual cleavage provides either a free acid or amide at the carboxyl terminus. Linkers are not resin-specific, and include peptide acids such as 4-hydroxymethylphenoxyacetyl-4′-methylbenzyhydrylamine (HMP), or peptide amides such as benzhydrylamine derivatives.

Following the preparation of the solid phase support with an appropriate solvent, the next step is to deprotect the amino acid to be attached to the peptide chain. Deprotection is carried out with a mild base treatment (picrodine or piperidine, for example) for temporary protective groups, while permanent side-chain protecting groups are removed by moderate acidolysis (trifluoroacetic acid, or TFA, as an example).

Following deprotection, the amino acid chain extension, or coupling, is characterized by the formation of peptide bonds. This process requires activation of the C-alpha-carboxyl group, which may be accomplished using one of five different techniques. These are, in no particular order, in situ reagents, preformed symmetrical anhydrides, active esters, acid halides, and urethane-protected N-carboxyanhydrides. The in situ method allows concurrent activation and coupling; the most popular type of coupling reagent is a carbodiimide derivative, such as N,N″-dicyclohexylcarbodiimide or N,N-diisopropylcarbodiimide.

After the desired sequence has been synthesized, the peptide is cleaved from the resin. This process depends on the sensitivity of the amino acid composition of the peptide and the side-chain protector groups. Generally, however, cleavage is carried out in an environment containing a plurality of scavenging agents to quench the reactive carbonium ions that originate from the protective groups and linkers. One common cleaving agent is TFA.

In short, summary SPPS requires the repetitive steps of deprotecting, activating, and coupling to add each acid, followed by the final step of cleavage to separate the completed peptide from the original solid support.

Two distinct disadvantages exist with respect to current SPPS technology. The first is the length of time necessary to synthesize a given peptide. Deprotection steps can take 30 minutes or more. Coupling each amino acid to the chain as described above requires about 45 minutes, the activation steps for each acid requires 15-20 minutes, and cleavage steps require two to four hours. Thus, synthesis of a mere twelve amino acid peptide may take up to 14 hours. To address this, alternative methods of peptide synthesis and coupling have been attempted using microwave technology. Microwave heating can be advantageous in a large variety of chemical reactions, including organic synthesis because microwaves tend to interact immediately and directly with compositions or solvents. Early workers reported simple coupling steps (but not full peptide synthesis) in a kitchen-type microwave oven. Such results are not easily reproducible, however, because of the limitations of a domestic microwave oven as a radiation source, a lack of power control, and reproducibility problems from oven to oven. Others have reported enhanced coupling rates using microwaves, but have concurrently generated high temperatures that tend to cause the solid phase support and the reaction mixtures to degenerate. Sample transfer between steps has also presented a disadvantage.

Another problem with the current technology is aggregation of the peptide sequence. Aggregation refers to the tendency of a growing peptide to fold back onto itself and form a loop, attaching via hydrogen bonding. This creates obvious problems with further chain extension. Theoretically, higher temperatures can reduce hydrogen bonding and thus reduce the fold-back problem, but such high temperatures can create their own disadvantages because they can negatively affect heat-sensitive peptide coupling reagents. For this reason, SPPS reactions are generally carried out at room temperature, leading to their characteristic extended reaction times.

### Positron Emission Tomography

Positron emission tomography (PET) has become a vital imaging modality in the diagnosis and treatment of disease, most notably cancer. Recently, targeted and marker-specific biomolecular vectors have seen dramatically increased research both in the laboratory and the clinic [5, 8, 9]. Several tissue-specific peptides have been successfully modified to create radiolabeled versions that show excellent selectivity for their respective targets. Amongst others, examples include ¹⁸F- and ⁶⁴Cu-labeled somatostatin receptor type 2 (sstr-2) [10, 11]¹⁸F/⁶⁴Cu-labeled gastrin-releasing peptide receptor (GRPr) [12-15], and ¹⁸F/⁶⁴ Cu-labeled glucagon-like peptide 1 receptor (GLP1-R) [16-18] agonists and antagonists. Furthermore, Neurokinin-1 receptor imaging agents have been developed for the detection and assessment of various health conditions, including glial tumors and particularly glioblastomas [19, 20].

Translation of targeted peptide sequences to targeted radiolabeled imaging agents can be time-consuming and often relies on serendipity rather than rational design. In certain cases, this is due to the limited number of chemical tools that are applicable as linkers to attach radiotracers to peptide sequences. The available reactions can require harsh conjugation conditions or sometimes do not meet chemoselectivity requirements. An alternative could be the use of the tetrazine/trans-cyclooctene Diels-Alder cycloaddition. Over the last five years, the potential of this reaction has been exploited as a tool for the in vitro and in vivo labeling of biologically relevant probes. This extremely fast and selective reaction was first used in living cells for the conjugation of tetrazine-labeled fluorophores to their trans-cyclooctene-labeled reporters of interest, using both extracellular (e.g. [21]) as well as intracellular markers [22]. More recent applications include the use of tetrazine/trans-cyclooctene reactions for the design of nanoparticles [23, 24] or detection of cancer biomarkers in human samples [25]. Most notably, tetrazines/trans-cyclooctenes can be used as bioorthogonal labels for radiotracers, which makes them especially useful for conjugation of short-lived PET tracers [4, 26].

Using tetrazines/trans-cyclooctenes for the radiolabeling of peptides does not only provide high reactivity and clean reaction products; indeed, these bioorthogonal functional groups are also applicable in aqueous, physiological buffers, tolerant to naturally-occurring amino acids and proceed rapidly at room temperature, even in dilute samples [27]. Their applicability as a radiolabeling tool was shown on an ¹⁸F-labeled small molecule model ([4, 26, 28]). Most importantly, tetrazines and trans-cyclooctenes can be used to purify crude radiolabeled mixtures without the use of chromatography-based techniques, making the isolation of peptides and peptidomimetics faster and more efficient [26]. With this in mind, it is envisioned, for example, that employing tetrazines, trans-cyclooctene, and amino acid chemistry for the rational and multiplexed design of a radiolabeled transferrin receptor imaging agent can allow visualization and detection of glioblastoma even if the blood brain barrier is still intact.

1. Development of Novel Blood Brain Barrier-Permeable Imaging Agents for Glioblastoma.

More than 50% of all persons diagnosed with brain tumors in the United States have glioblastomas, which have a very poor prognosis. Despite major research efforts and progress in neuroimaging, neurosurgery, radiology and medical oncology, the overall survival of patients with this disease has changed little over the past 30 years [29]. One of the obstacles in early diagnosis and treatment of glioblastoma is the blood brain barrier, which can prevent targeting of malignant tissues and selective accumulation of imaging agents, especially in early stage and low-grade glioblastomas. Based on recently introduced transferrin receptor targeting peptides [30], a library of radiolabeled analogs and evaluate their ability to permeate the blood brain barrier and selectively accumulate in malignant tissues will be designed.

2. Development of Selective Modular Strategies for the Incorporation of Tetrazines and Trans-Cyclooctenes Via Automated Peptide Synthesizers.

One of the most exciting and recent approaches for biomedical imaging has been the use of the extremely rapid tetrazine/trans-cyclooctene reaction for radiotracer development, purification and in vivo imaging [4, 26, 28]. Given the unique applicability of this technology for the design of peptide based tracers, the development of a method of producing tetrazine/trans-cyclooctene amino acids and their incorporation into peptides for the development of peptide libraries featuring these amino acids, using standard peptide synthesizers is described, herein.

3. Multiplexed Screening Technologies for Peptide-Based PET Imaging Probes.

Due to low labeling specificity, selectivity and the lack of suitable attachment protocols, the development and evaluation of novel radiopharmaceuticals often occurs sequentially rather than parallelized. Herein the tetrazine/trans-cyclooctene peptides and their radiolabeled counterparts are used through development of combinatorial parallelized synthesis and semi-automated cell based screening protocols for the discovery of novel high affinity radiopharmaceuticals (n=˜14 (peptides)×6 (radiolabels)=(84 different candidates)). This will establish a novel methodology, in which potential radiolabeled peptides can be tested in a shorter amount of time, thus bringing promising candidates faster into animals and ultimately to the bedside.

### Preliminary Data

a. Development of Tetrazine/Trans-Cyclooctene Probes for ⁸⁹Zr, ⁶⁴Cu and ¹⁸F.

To date, it has been shown in multiple studies [3-5, 26, 28, 31, 32], that the tetrazine/trans-cyclooctene reaction is ideally suited to facilitate rapid and selective coordination of radiolabeled precursors and targeted probes under mild conditions and at low concentrations. Particularly, a ¹⁸F-trans-cyclooctene has been designed in 2011 [4], which has since shown its broad applicability as a toolkit-compound in three additional studies [26, 28, 31]. Additionally, tetrazine-conjugated ⁸⁹Zr and ⁶⁴Cu chelators, have been designed which were employed to selectively tag antibodies [5].

b. Design of Tetrazine/Trans-Cyclooctene Based Purification Technologies.

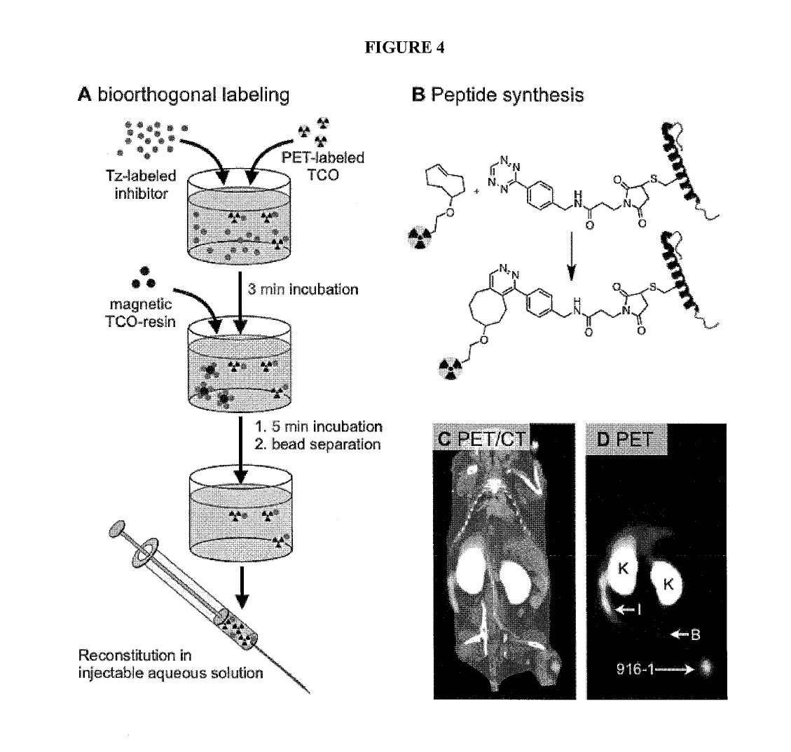

One of the major challenges for the production of high specific activity radiopharmaceuticals is the removal of non-radiolabeled precursors. Although for small molecules, removal of unwanted precursors (e.g. via techniques such as HPLC) can be relatively straightforward, it can represent a major pitfall for peptides, peptidomimetics and large biomolecules. Here, tetrazines/trans-cyclooctenes present the unique opportunity to remove unwanted starting material by selective and specific immobilization on macromolecules or nanoparticles (FIG. 4A). This technique was first introduced in 2011 [26]. It has since been applied for multiple projects [28, 31].

c. Bioorthogonal Labeling of a 39 Amino Acid Artificial Peptide.

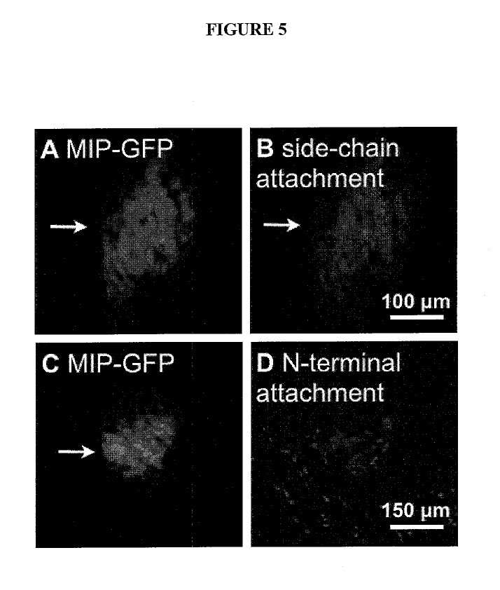

In previous studies, it was shown that even for long peptides (39 amino acids) with sub-nanomolar binding (˜1 nM), changes in the attachment point of reporters can have significant effects not only on target binding, but also to the pharmacological profile (FIG. 5A-D) [33, 34]. This underscores the necessity of developing a stable, rational, and modular approach that allows selective incorporation at a specific position within the side-chain of a protein, irrespective of other functional groups present in the biomolecule. Furthermore, it was confirmed that tetrazines/trans-cyclooctenes are inert to naturally occurring amino acids, eliminating the possibility of cross-reactivity. Using a two step labeling strategy (FIG. 4B), it was demonstrated that tetrazines/trans-cyclooctenes do not get metabolized rapidly in vivo, and that they can be used for quantitative site-selective labeling of peptides (FIG. 4C and FIG. 4D) [31].

### Methods and Strategies—Part 1

1. Rationale—Tetrazines and trans-cyclooctenes are valuable tools for the radiolabeling of peptides and peptidomimetics. To date, though, all applications require the use of an additional reaction to introduce either tetrazine or trans-cyclooctene into the peptide (e.g. succinimide esters, maleimides/cysteines). This additional step increases development time for radiopharmaceuticals, makes additional purification necessary and has limitations regarding the backbone sites at which such tetrazines/trans-cyclooctenes can be incorporated.

2. It is believed that the incorporation of tetrazine and trans-cyclooctene artificial amino acids into peptides via peptide synthesizers will allow fast, selective and rational development of radiolabels with high specific activity.

3. Materials and Methods

Synthesis of Fmoc- and Boc-Protected Tetrazines and Trans-Cyclooctene Amino Acids for Solid Phase Peptide Synthesis (SPPS):

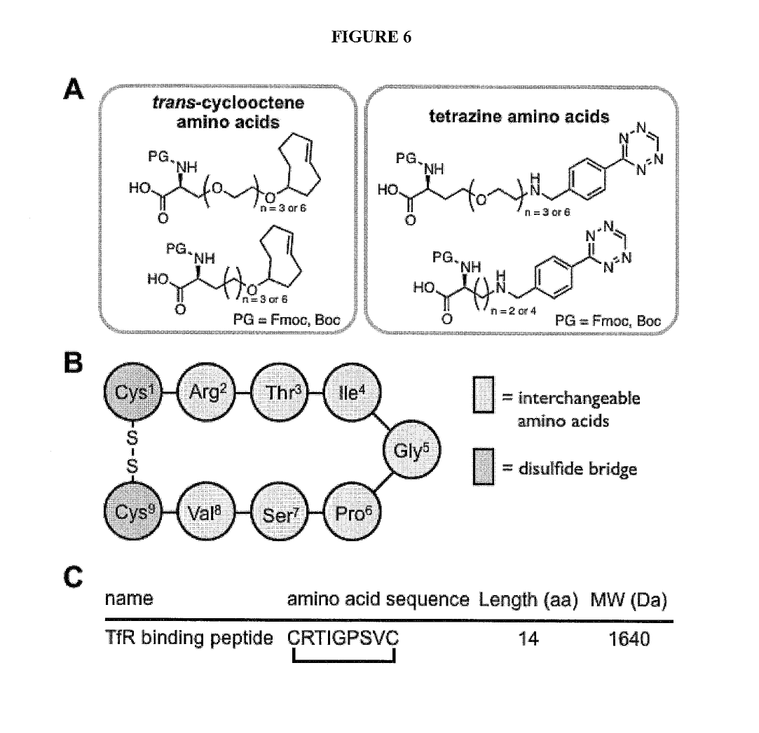

A small library of tetrazine- and trans-cyclooctene amino acids (2 representatives for each reactive group) suitable for solid phase peptide synthesis (SPPS) will be synthesized. Their side-chains will be linked to the amino acids via either short alkyl chains (2 or 4 CH₂-groups) or ethylene glycol units (3 or 6 glycol units). All novel artificial amino acids will be synthesized with both Fmoc- and Boc-protective groups (see FIG. 6A).

Stability Studies of Monomeric Amino Acids:

Both tetrazines and trans-cyclooctenes are stable to a wide range of solvents and conditions typically employed in bioconjugation reactions. They are also stable to peptide coupling conditions as well as the deprotection conditions of various protective groups. Both reactive groups have been used successfully without any detectable decomposition under peptide coupling conditions (using peptide coupling reagents as diverse as HBTU/HOBt/DIPEA, DIC/DIPEA, EDCl/Et₃N, and DCC/DMAP). Since solid phase peptide synthesis requires multiple coupling as well as deprotection steps, the stability profiles of both tetrazines as well as trans-cyclooctenes will be carefully determined, taking into account all necessary deprotection conditions, for example a) 20-50% piperidine in DMF (Fmoc-deprotection; b) 10-50% TFA in DMF (final cleavage from resin using an Fmoc-strategy as well as deprotection of Boc-protected side chains); c) anhydrous HF or TFMSA (final cleavage from resin using a Boc-strategy); d) HBr/acetic acid (Cbz-deprotection); e) [(PPh₃)₄Pd], acetic acid, N-methyl morpholine (Alloc-deprotection). The effects of prolonged exposure to coupling reagents, e.g. HBTU/HOBt/DIPEA, DIC/DIPEA, EDCl/Et₃N, and DCC/DMAP will be further evaluated. The synthesized tetrazines and trans-cyclooctenes will be deemed “stable” towards a particular condition if the total decomposition of one such amino acid after being exposed 10 times longer the standard amount of time is less than 5% (<0.5% decomposition for each exposure). It is envisioned, that a Boc-based protection strategy will likely be most successful for tetrazine-containing peptides, whereas Fmoc-mediated solid phase peptide synthesis is more likely to be successful for trans-cyclooctene containing peptides.

Incorporation of Tetrazines/Trans-Cyclooctenes in Libraries of Peptides (n=˜100):

The goal is to establish tetrazine/trans-cyclooctene amino acids as platform chemicals for the combinatorial synthesis of radiopharmaceuticals.

Transferrin-Receptor Binding Protein:

As a target sequence, CRTIGPSVC (SEQ ID No:7) will be used, a peptide which was shown to bind to the transferrin-receptor (FIG. 6B-FIG. 6C) [30]. This peptide can cross the blood brain barrier and accumulates preferentially in glioblastomas (based on the increased expression of transferrin-receptors). The 9 amino acid peptide displays an ideal model system for tetrazine/trans-cyclooctene amino acids: a) its length (9 amino acids) is ideal for a peptide-based radiopharmaceutical (similar to ¹¹¹1 n-bombesin and ¹¹¹In-pentetreotide, which both have 8 amino acids); b) the peptide displays primary amines (N-terminus), alcohols (Thr³, Ser⁷), carboxylic acids (C-terminus) as well two thiols (Cys¹, Cys⁹) which need to be oxidized to a disulfide bridge. Tetrazine amino acids and trans-cyclooctene amino acids will be used and analogs of the target peptide synthesized, where all non-cysteine amino acids are sequentially exchanged, yielding 7×2=14 different novel bioorthogonal analogs, seven of which are containing tetrazines, and seven containing trans-cyclooctenes. Negative controls will be synthesized in which (in addition to the tetrazines/trans-cyclooctenes) four or more amino acids are exchanged with glycine. This will yield analogs with reduced or no selectivity for the transferrin-receptor.

Somatostatin-14:

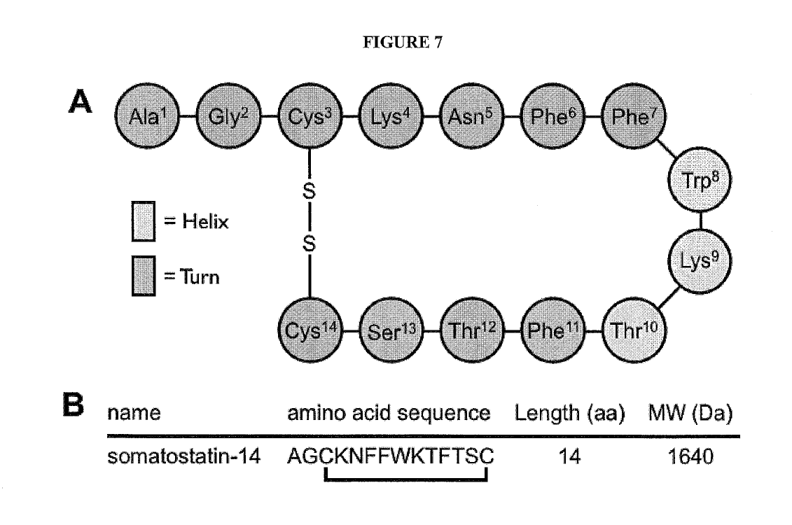

To confirm that these novel agents can be used in standard solid phase peptide synthesis, the 14 amino acid peptide somatostatin-14, AGCKNFFWKTFTSC (SEQ ID No:8), will serve as a model system (FIG. 7 A-B). This peptide was chosen because it displays all functional groups and features which might be a challenge for the incorporation of tetrazine/trans-cyclooctene amino acids as building blocks: a) its length (14 amino acids) is longer than most established radiopharmaceuticals (e.g. ¹¹¹In-bombesin and ¹¹¹In-pentetreotide); b) the peptide displays amines (N-terminus, Lys⁴), alcohols (Ser¹³), carboxylic acids (C-terminus) as well two thiols (Cys³, Cys¹⁴) which need to be oxidized to a disulfide bridge. Four different tetrazine amino acids and four different trans-cyclooctene amino acids will used and analogs of somatostatin synthesized, where all non-cysteine amino acids are sequentially exchanged, yielding 8×12=96 different novel bioorthogonal somatostatin analogs, 44 of which are containing tetrazines, and 44 containing trans-cyclooctenes. Negative controls in which (in addition to the tetrazines/trans-cyclooctenes) four or more amino acids are exchanged with glycine will be synthesized. This will yield peptides with reduced to no selectivity for the somatostatin-receptor.

4. Data analysis and statistics. All synthesized tetrazine and trans-cyclooctene amino acids will be analyzed using standard techniques, including ¹H- and ¹³C-NMR (both one-dimensional as well as two-dimensional), LC/MS and HRMS. All stability measurements will be conducted at least in triplicate, using internal standards, and statistical analysis will be employed to determine the results. Peptides will be analyzed using HRMS measurements, MALDI-MS spectra as well as LC/MS analysis. All small molecules will be purified by either silica column chromatography or reversed-phase HPLC. Peptides will be purified via reversed-phase HPLC.

5. Impact, Pitfalls, and Alternatives. This aim will confirm the applicability of tetrazines/trans-cyclooctenes in solid phase peptide synthesis. This proof-of-principle will have profound impact on the future design of peptide-based radiopharmaceuticals. The design of novel peptides will become faster, more rational and modular. Although unlikely, it is possible that the stability of tetrazines/trans-cyclooctenes is insufficient under standard peptide coupling conditions. Stability will be deemed insufficient if the total decomposition of one such tetrazine/trans-cyclooctene amino acid exceeds 5% after being exposed 10 times to standard coupling conditions (>0.5% decomposition for each exposure). If this is the case, coupling conditions will be improved by using ultra-pure reagents (sodium-dried DMF, dry coupling reagents (benzene-lyophilization or sublimation, argon-atmosphere)). Alternatively, another approach can be the assembly of peptide fragments using solution-phase peptide synthesis (which will reduce the amount of time tetrazines/trans-cyclooctenes are exposed to coupling reagents). If no alternative route allows clean and selective assembly of transferrin-binding analogs, alternative bioorthogonal catalyst-free reaction pairs will be considered (e.g. azide/DIFO, azide/DBCO; [35]).

### Methods and Strategies—Part 2

1. Rationale—The development of novel radiolabeled peptides and peptidomimetics is usually slow, with most research projects focusing on just 1-3 different radiolabeled candidates. In contrast, standard peptide synthesis allows the generation of orders of magnitude higher candidate peptides in a short amount of time. The translation of such a peptide-library to their corresponding radiolabeled imaging candidates is limited by a) the time needed to develop labeling techniques for the particular peptide; b) the selectivity of labeling reactions and c) the need to isolate each radiolabeled peptide by HPLC, size exclusion chromatography, or affinity chromatography. The goal of the following experiments is to establish a routine protocol, in which tetrazine or trans-cyclooctene containing peptides from Part 1 can be reacted with their bioorthogonally radiolabeled counterparts (e.g. tetrazine-⁸⁹Zr-DFO/trans-cyclooctene-⁸⁹Zr-DFO), yielding a small library of radiolabeled peptides in high purity and high specific activity which can then be used and evaluated without sequential chromatographic purification of their individual representatives.

2. Hypothesis—the multiplexed synthesis of radiolabeled peptides will allow high throughput development of novel targeted probes and ultimately accelerated translation to clinical research.

3. Materials and Methods

Synthesis of Tetrazine and Trans-Cyclooctene Labeled Chelators:

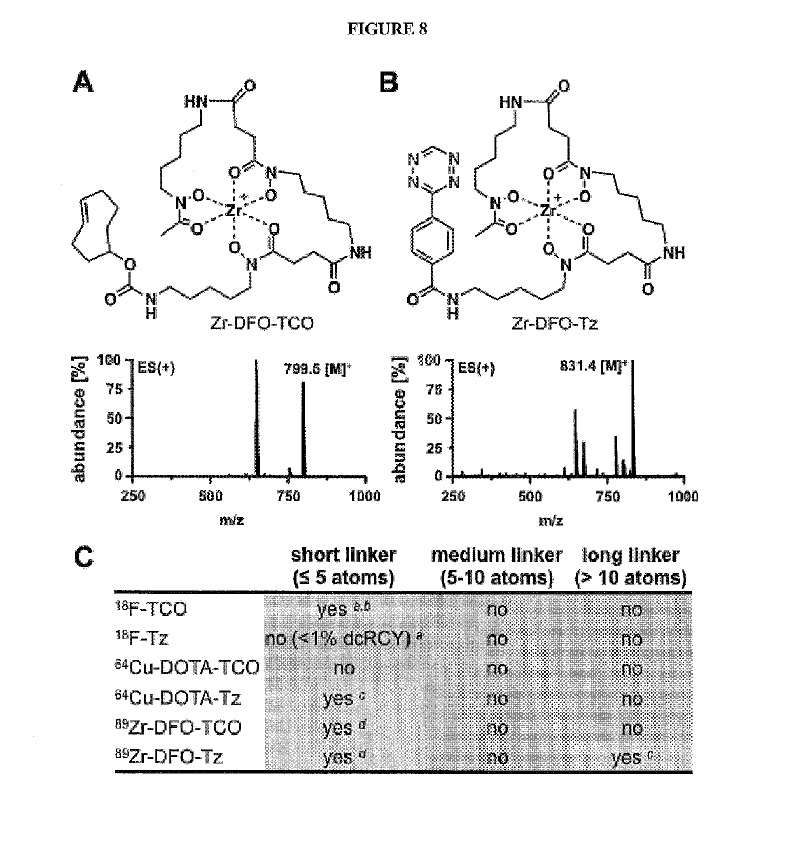

Based on their selectivity, specificity and tolerance toward naturally occurring functional groups, bioorthogonal tetrazines and trans-cyclooctene pairs have proven to be valuable tools for the labeling of peptides. To date, three types of radiolabeled tetrazine/trans-cyclooctene linkers are identified in the literature: ¹⁸F-trans-cyclooctene, ⁸⁹Zr-DFO-tetrazine and ⁶⁴Cu-DOTA-tetrazine ([4, 5]). Others were prepared in-house (FIG. 8A-B). Based on existing methodologies, the portfolio of available linkers and precursors for ¹⁸F, ⁸⁹Zr, and ⁶⁴Cu (three tetrazines and trans-cyclooctenes for each label) will be expanded by modulating their linker lengths and thus solubilities (FIG. 8B). This will allow for the tuning of the blood half-life of radiolabeled peptides and the reduction in (or of) the likelihood of steric hindrance/perturbation of binding affinity. For each precursor/chelator, labeling methodologies will be developed and optimized (for ¹⁸F: dcRCY >80%, RCP >98%, >1.5 mCi yield; for ⁶⁴Cu/⁸⁹Zr: dcRCY >95%, RCP >98%, >1.0 mCi yield).

Multiplexed Generation and Purification of Radiolabeled Peptides:

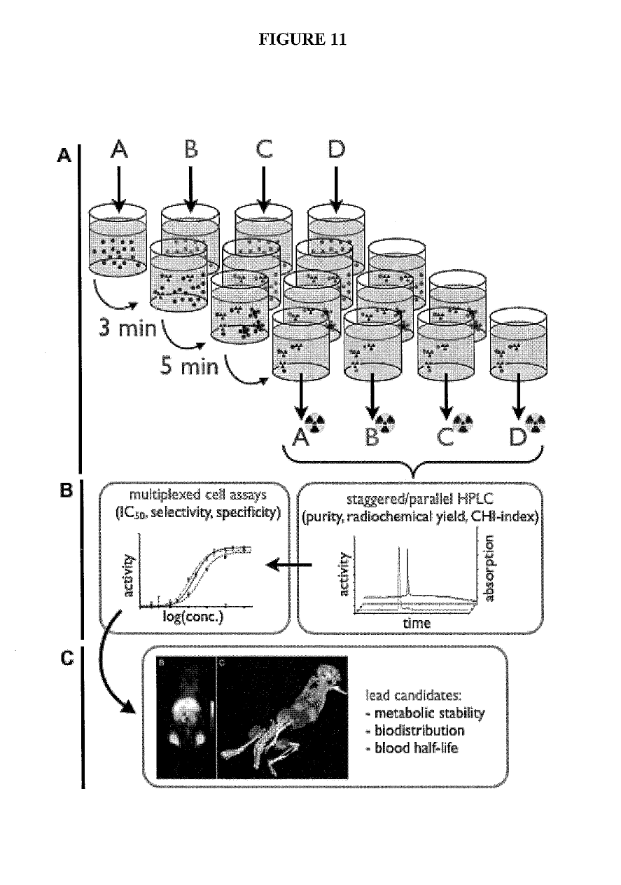

Based on methods for small molecules, a library of tetrazine/trans-cyclooctene labeled transferrin-binding peptides (aim 1, one peptide/well, n(total)=14) will be incubated with a sub-stoichiometric amount of their radiolabeled bioorthogonal counterparts in aqueous buffered solutions. After the incubation (5 minutes), scavenger-resins will be added to remove unreacted excess cold material (incubation time=5 minutes) and the resulting purified high specific activity peptides filtered (FIG. 11). Using a staggered/parallel analytical HPLC-method, the radiochemical purity and specific activity of each peptide will be determined (FIG. 11). HPLC will also allow determination of the chromatographic hydrophobicity index of radiolabeled peptides (a measure of lipophilicity and log P/log D [36]). This data will be further validated by mathematical estimation of the log P values.

Cell-Based Selectivity Screening of Radiolabeled Peptides:

Using a parallelized 96-well based setup, the selectivity of the hot radiolabeled peptides towards transferrin-receptor positive cells (e.g. U87-MG) will be determined. 100 nM solutions of the peptides will be incubated with the cells in the presence and absence of a known transferrin-receptor antagonist (transferrin, 5 μM). Radiolabeled peptides which allow uptake reduction ≧95% will be further investigated in a radioactive binding assay (concentrations ranging from 0.001 nM to 100 nM), with and without addition of transferrin (5 μM, 0.5 μM and 0 μM). Promising candidates (IC₅₀≦10 nM) will also be screened against transferrin receptor negative cell lines.

Validation of Screening Results Using Validated Gold Standards:

Somatostatin-analogs are ideal peptide models for this proof-of principle screening study because there are fast and reliable non-radioactive screening assays available to determine IC₅₀ values. All peptides will be re-synthesized (aim 1, one peptide/well, n(total)˜100) using cold bioorthogonal counterparts (¹⁹F, Cu, Zr) and subject them to a fluorometric imaging plate reader assay. The assay will be based on the activation of calcium flux in sstr-2 expressing cell lines by somatostatin-analogs. Commercially available sstr-2 expressing Chem-1 cell lines (Millipore) will be used and their calcium-flux determined by fluorescence measurements using a calcium sensitive dye (Fluo-8 No Wash Ca²⁺, Abeam), similar to procedures established before [37].

Translation of Hit Candidates to Animal Tumor Models: Transferrin-Receptor Binding Protein:

Promising candidates (IC₅₀≦10 nM, specific uptake ≧95%) will be tested in animal models of glioblastomas (using either orthotopic [38] or subcutaneous glioblastoma xenografts). For glioblastoma xenografts, the tumor cell lines will be injected subcutaneously into BALB/c mice and the uptake of radiolabeled transferrin receptor binding peptides tested once the tumors reached diameters of ˜5 mm. Biodistribution data will be obtained using standard γ-counters, while pharmacokinetics will be determined using dynamic PET/CT scans. Somatostatin-14: Hit candidates (IC50≦10 nM, specific uptake ≧95%) will be tested in animal models of pancreatic tumor xenografts. AR42J (sstr-2 positive, [39]) and HCT116 (sstr-2 negative) will be injected subcutaneously into BALB/c mice and the uptake of radiolabeled somatostatin-analogs tested once the tumors reached diameters of ˜5 mm. Biodistribution data will be obtained using standard PET γ-counters while pharmacokinetics will be determined using dynamic PET/CT scans.

4. Data analysis and statistics. Radiolabeling of peptides will be conducted in triplicate, and statistical analysis will be employed to determine the derived specific uptake, radiochemical purity and Log P values. Cell uptake experiments (hot and cold) will be conducted in triplicate, and statistical analysis will be used to confirm there is no statistically significant difference between results obtained from hot (¹⁹F, ⁶⁴Cu, ⁸⁹Zr) and cold (¹⁹F, Cu, Zr) TfR binding peptide analogs. For biodistribution and pharmacokinetic experiments, automated image processing/automated analysis will be used where possible to eliminate human bias. Positive and negative controls will be added where appropriate.

5. Impact, Pitfalls, and Alternatives. The successful combination of bioorthogonal tetrazine/trans-cyclooctene chemistry and its application in parallelized screening of PET imaging agents will lay the groundwork for the fast, rational, multiplexed development of radiolabeled targeted peptides. This aim will provide the scientific community with a toolkit, which can easily be translated toward novel and emerging targets. Although not expected, it is possible that this proof-of-principle screening will not yield radiolabeled hit-candidates with sufficiently high receptor affinity, selectivity and sufficiently good pharmacokinetics. If this is the case, the screening will alternate to other peptidic scaffolds relevant for brain tumor research or more and better validated peptidic scaffolds. Examples of peptidic scaffolds relevant for brain tumor research include somatostatin analogs (astrocytoma, medulloblastoma and neuroblastoma) or substance P analogs (glial tumors and glioblastomas) [30]. If no alternative peptide scaffold allows high affinity peptide analogs with excellent pharmacokinetics, alternative bioorthogonal catalyst-free reaction pairs will be considered (e.g. azide/DIFO, azide/DBCO). Examples of validated peptidic scaffolds include Octerotide (8 amino acids, cyclic), Lanreotide (8 amino acids, cyclic) or α-MSH/β-MSH (14 and 22 amino acids, respectively, both linear). If no alternative peptide scaffold allows high affinity peptide analogs with excellent pharmacokinetics, alternative bioorthogonal catalyst-free reaction pairs will be considered (e.g. azide/DIFO, azide/DBCO).

### EXPERIMENTAL METHODS

Materials.

Unless otherwise noted, all reagents were purchased from Sigma-Aldrich (St. Louis, Mo.) and used without further purification. Boc-Gly Merrifield resin and Boc-Lys-OMe were purchased from Bachem (Torrance, Calif.). Boc-Ala-OH, Boc-Lys-OH, Boc-Phe-OH and Boc-Val-OH were purchased from Novabiochem (Merck KGaA, Darmstadt, Germany). 3-(4-phenylacetic acid)-1,2,4,5-tetrazine (Tz-COOH) and (E)-cyclooct-4-enyl 2,5-dioxopyrrolidin-1-yl carbonate (TCO-NHS) were synthesized as described before [40, 41]. HPLC analyses were performed using a Shimadzu HPLC equipped with 2 LC-10AT pumps, a SPD-M10AVP photodiode array detector, a Flow Count PIN diode radiodetector from BioScan and a Waters Atlantis T3 6×250 mm, 5 um column. Gradients of 95:5 H2O/AcN with 0.1% TFA to 0:100 H₂O/AcN over 18 min, 1 mL/min flowrate were used. For preparative HPLCs, a Phenomenex Jupiter 250×100 mm, 5 μm, and flowrates of 5 mL/min were used. Radioactivity measurements were performed with a Capintec CRC1243 Dose Calibrator (Capintec, Ramsay, N.J., USA). For TLC measurements with radioactive materials, a Bioscan AR2000 (Bioscan, Inc., Washington, D.C.) was used. Microwave irradiations were carried out using a Biotage Initiator microwave synthesizer (Biotage, LLC, Charlotte, N.C.). Low resolution mass spectra (LRMS) were recorded with a Waters Acquity UPLC with electrospray ionization SQ detector (ESI). High resolution mass spectra (HRMS) were recorded with a Waters LCT Premier system (ESI). Proton nuclear magnetic resonance (¹H NMR) spectra were recorded on a Bruker AVIII (500 MHz) spectrometer. Chemical shifts for protons are reported in parts per million (ppm) and are referenced against the residual proton resonance of deuterated solvents (chloroform-d: ¹H, 7.26 ppm; methanol-d₄: ¹H, 3.31 ppm; dimethylsulfoxide-d₆: ¹H, 2.50 ppm). NMR data are reported as follows: chemical shift, multiplicity (s=singlet, d=doublet, t=triplet, m=multiplet), coupling constants (Hz) and integration.

### Synthesis of 3-(4-phenylacetic acid)-1,2,4,5-tetrazine succinimidyl ester (Tz-NHS)

N-Hydroxysuccinimide (163 mg, 1.42 mmol) and triethylamine (495 μL, 3.55 mmol) were added to a mixture of 3-(4-phenylacetic acid)-1,2,4,5-tetrazine (150 mg, 0.71 mmol) in dichloromethane (15 mL) and the reaction mixture stirred for 6 hours at room temperature. The mixture was extracted with acetic acid (1M, 2×10 mL) and water (2×10 mL), dried (MgSO₄) and volatiles removed in vacuo. The crude product was purified by column chromatography (DCM/MeOH=97.5/2.5), yielding the title compound as a pink solid (67.3 mg, 0.22 mmol, 31%). ¹H NMR (500 MHz, methanol-d₄) δ=10.60 (s, 1H), 8.51 (d, ³J_{HH}=8.3, 2H), 7.55 (d, ³J_{HH}=8.3, 2H), 7.20 (d, ³J_{HH}=7.7, 1H), 4.32 (s, 2H), 2.36 (s, 4H); LC-ESI-MS(−) m/z (%)=313.1 [M−H]⁻ (100), 312.1 [2M−H]⁻ (100).

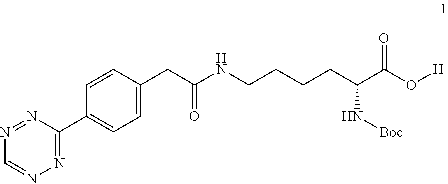 [SMILES: CC(C)(C)OC(=O)N[C@H](CCCCNC(=O)Cc1ccc(-c2nncnn2)cc1)C(=O)O]

### Synthesis of Boc-τ-OH 1

3-(4-phenylacetic acid)-1,2,4,5-tetrazine succinimidyl ester (55.0 mg, 0.18 mmol) was dissolved in a mixture of Boc-Lys-OH (53.2 mg, 0.36 mmol) and triethylamine (63 μL, 0.45 mmol) in methanol (6 mL) and stirred for 2 h. The reaction mixture was dried under reduced pressure and the resulting crude product purified by column chromatography (5%-25% MeOH in DCM), yielding the title compound as a pink film (40.3 mg, 0.09 mmol, 50%). ¹H NMR (500 MHz, methanol-d₄) δ=10.31 (s, 1H), 8.55 (d, ³J_{HH}=8.4, 2H), 7.56 (d, ³J_{HH}=8.4, 2H), 4.05 (dd, ³J_{HH}=8.7, ³J_{HH}=4.7, 1H), 3.22 (t, ³J_{HH}=6.9, 2H), 1.86-1.61 (m, 2H), 1.58-1.38 (m, 13H); LC-ESI-MS(+) m/z (%)=467.3 [M+Na]⁺ (30), 911.6 [2M+Na]⁺ (10); LC-ESI-MS(−) m/z (%)=443.3 [M−H]⁻ (10), 887.6 [2M−H]⁻ (20); HRMS-ESI m/z calcd. for [C₂₁H₂₈N₆O₅Na]⁺ 467.2005. found 467.2019 [M+Na]⁺.

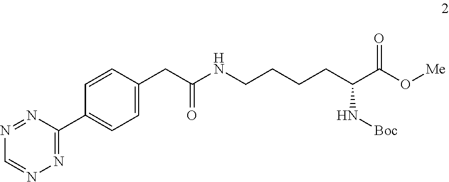 [SMILES: COC(=O)[C@@H](CCCCNC(=O)Cc1ccc(-c2nncnn2)cc1)NC(=O)OC(C)(C)C]

### Synthesis of Boc-τ-OMe 2

N-(3-Dimethylaminopropyl)-N′-ethylcarbodiimide hydrochloride (51.8 mg, 0.27 mol) was added to a mixture of 3-(4-phenylacetic acid)-1,2,4,5-tetrazine succinimidyl ester (20 mg, 0.09 mmol), Boc-Lys-OMe (28.6 mg, 0.11 mmol), triethylamine (63 μL, 0.45 mmol) in dichloromethane (5 mL) and the reaction mixture stirred over night at room temperature, before it was dried under reduced pressure and the resulting crude product purified by column chromatography (5% MeOH in DCM), yielding the title compound as a pink film (20.8 mg, 0.05 mmol, 56%). ¹H NMR (500 MHz, dimethylsulfoxide-d₆) δ=10.57 (s, 1H), 8.44 (d, ³J_{HH}=8.2, 2H), 8.13 (t, ³J_{HH}=5.5, 1H), 7.55 (d, ³J_{HH}=8.2, 2H), 7.20 (d, ³J_{HH}=7.7, 1H), 3.91 (m, 1H), 3.61 (s, 3H), 3.55 (s, 2H), 3.05 (q, ³J_{HH}=6.5, 2H), 1.65-1.52 (m, 2H), 1.43-1.27 (m, 13H); LC-ESI-MS(+) m/z (%)=359.3 [M−Boc+H]⁺ (75), 459.3 [M+H]⁺ (40), 917.7 [2M+H]⁺ (30); HRMS-ESI m/z calcd. for [C₂₂H₃₀H₆O₅Na]⁺ 481.2171. found 481.2175 [M+H]⁺.

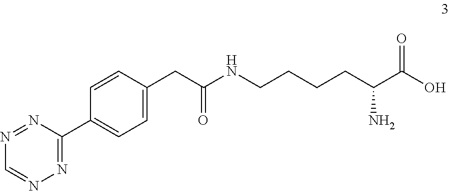 [SMILES: N[C@H](CCCCNC(=O)Cc1ccc(-c2nncnn2)cc1)C(=O)O]

### Synthesis of H-τ-OH 3

Boc-τ-OH 1 (10 mg, 22.5 μmol) was dissolved in a mixture of TFA/DCM=1/1 and vigorously stirred for 1.5 hours, before volatiles were removed in vacuo and the resulting crude material purified via HPLC, yielding the title compound as a pink solid (5.6 mg, 16.3 μmol, 72%). ¹H NMR (500 MHz, methanol-d₄) δ=10.32 (s, 1H), 8.55 (d, ³J_{HH}=8.3, 2H), 7.57 (d, ³J_{HH}=8.3, 2H), 3.92 (t, ³J_{HH}=6.3, 2H), 3.65 (s 2H), 3.24 (dt, ³J_{HH}=6.3, ³J_{HH}=3.2, 2H), 2.00-1.84 (m, 2H), 1.62-1.42 (m, 2H); LC-ESI-MS(+) m/z (%)=345.2 [M+H]⁺ (100), 367.2 [M+Na]⁺ (15); HRMS-ESI m/z calcd. for [C₁₆H₂₁N₆O₃]⁺ 345.1668. found 345.1675 [M+H]⁺.

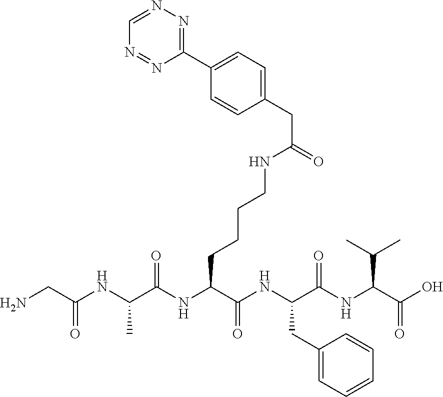 [SMILES: CC(C)[C@H](NC(=O)[C@H](Cc1ccccc1)NC(=O)[C@H](CCCCNC(=O)Cc1ccc(-c2nncnn2)cc1)NC(=O)[C@H](C)NC(=O)CN)C(=O)O]

### Synthesis of GAτFV 4

The synthesis was carried out on a 0.01-0.02 mmol scale in a fritted syringe (3 mL volume), using a Merrifield-resin which was pre-loaded with Boc-G-OH (20 mg, 0.5-1.0 mmol/g). The resin was swollen by washing with DMF (2×2 mL) and DCM (2×2 mL). N^{α}-Boc groups were removed by addition of a solution of TFA and DCM (TFA/DCM=1/1; 1×5 min, 2 mL and 1×20 min, 2 mL), followed by washing with DCM (2×2 mL) and DMF (2×2 mL) Amino acids were coupled (1 h, RT) without prior neutralization of the resin, using a freshly prepared mixture of BOP (0.04 mmol, 18 mg) DIPEA (0.12 mmol, 16 mg) and amino acid (0.08 mmol) in DMF (2.5 mL). After coupling, the resin was washed with DMF (3×2 mL) and DCM (3×2 mL). After completion of the deprotection/coupling sequence, the peptide was transferred to a microwave vessel, suspended in a solution of TFA/H₂O (TFA/H₂O=97.5/2.5, 500 μL), and microwaved (2×5 min, 50 Hz). The resin was removed by filtration and 10 mL diethylether added to the supernatant. The resulting crude precipitate was filtered off and purified via HPLC to yield the title compound as a pink solid (0.9 mg, 1.3 μmol, 7% yield). LC-ESI-MS(+) m/z (%)=719.5 [M+H]⁺ (20); LC-ESI-MS(−) m/z (%)=717.5 [M−H]⁻ (10); HRMS-ESI m/z calcd. for [C₃₅H₄₇N₁₀O₇]⁺ 719.3633. found 719.3629 [M+H]⁺.

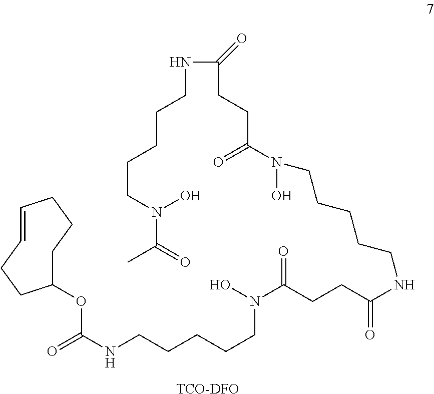

### Synthesis of TCO-DFO 7

(E)-cyclooct-4-enyl 2,5-dioxopyrrolidin-1-yl carbonate was added to a solution deferoxamine mesylate salt (20 mg, 30 μmol) and triethylamine (21 μL, 0.15 mmol) were dissolved in solution of acetonitrile/water (AcN/H₂O=1/1, 1 mL) and stirred at room temperature for 90 minutes, before volatiles were removed under reduced pressure and the crude product purified via HPLC, yielding the title compound as a white solid (6.6 mg, 9 μmol, 30%). ¹H NMR (500 MHz, dimethylsulfoxide-d₆) δ=9.61 (m, 2H), 7.76 (m, 1H), 6.90 (m, 1H), 5.60-5.40 (m, 2H), 4.21-4.17 (m, 1H), 3.44 (m, 6H), 3.03-2.87 (m, 6H), 2.61-2.53 (m, 4H), 2.29-2.21 (m, 7H), 1.96 (s, 3H), 1.91-1.85 (m, 4H), 1.56-1.15 (m, 22H); LC-ESI-MS(+) m/z (%)=357.4 [M+2H]²⁺ (10), 713.6 [M+H]⁺ (100), 735.5 [M+Na]⁺ (70), 759.4 [M+HCOO]⁺ (10); HRMS-ESI m/z calcd. for [C₃₄H₆₁N₆O₁₀]⁺ 713.4474. found 713.4449 [M+H]⁺.

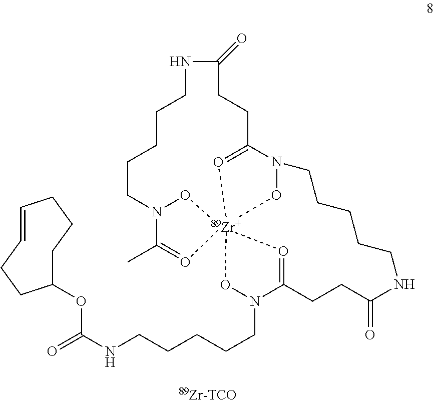

### Radiochemical Synthesis of ⁸⁹Zr-TCO 8

[⁸⁹Zr]Zr-oxalate (59.2-74 MBq, 1.600-2.000 μCi) in oxalic acid (1.0 M, 150 μL) was adjusted to pH 7.2-8.5 with Na₂CO₃ (1.0 M, approx. 150 μL). After the evolution of CO₂(g) stops, the ⁸⁹Zr was added to a solution of TCO-DFO in PBS/DMSO (1 mM, 100 uL, PBS/DMSO=9/1). The reaction solution was stirred at 37° C. for 1 hour, before the reaction progress was assayed using ITLC with an eluent of 1 M citric acid. In the ITLC experiments, ⁸⁹Zr-TCO 8 remains at the baseline, while ⁸⁹Zr⁴⁺ ions and other [⁸⁹Zr]-complexes elute with high Rf-values. The crude reaction mixture was purified using HPLC, and volatiles were removed in vacuo, yielding the title compound ⁸⁹Zr-TCO 8 with >99% radiochemical purity (39% uncorrected isolated RCY, and a specific activity of >5.59 mCi/mol or >6.98 mCi/mg).

### Radiochemical Synthesis of ⁸⁹Zr-GAτFV 9

GAτFV 4 (20 nMol, 20 μl, 1 mM in DMSO) was added to ⁸⁹Zr-TCO (56 μCi, 10 μL, 5.56 mCi/mL in DMSO) and the mixture agitated at room temperature for 20 minutes, before the ⁸⁹Zr-GAτEV 9 was purified via HPLC, yielding the title compound in >99% purity (95% uncorrected isolated RCY, and a specific activity of >3.00 mCi/μmol or >3.75 mCi/mg).

Determination of Tetrazine-Stability Under Boc-Deprotection Conditions.

Trifluoroacetic acid (760 μL) was added to a solution of of H-τ-OH 3 (5 mM, 20 μL, DMSO) and coumarin (5 mM, 20 μL, DMSO) and the resulting mixture stirred at room temperature. A control sample consisted of dimethyl sulfoxide (760 which was added to a solution of of H-τ-OH 3 (5 mM, 20 μL, DMSO) and coumarin (5 mM, 20 μL, DMSO). The amount of H-τ-OH 3 relative to coumarin was measured at 0, 0.5, 1 h, 2 h, 3 h, 4 h, 5 h, 6 h, 7 h, 8 h and 9 h using HPLC-analysis and the results compared to the control sample.

Determination of Tetrazine-Stability Under Coupling Conditions.

Solutions of BOP (10 mM, 25 μL, DMF) and DIEA (100 mM, 25 μL, DMF) were added to a solution of of Boc-τ-OMe 2 (5 mM, 25 μL, DMF) and coumarin (5 mM, 25 μL, DMF). A control sample consisted of dimethylformamide (50 μL), which was added to a solution of of Boc-τ-OMe 2 (5 mM, 25 μL, DMF) and coumarin (5 mM, 25 μL, DMF). The amount of Boc-τ-OMe 3 relative to coumarin was measured at 0, 0.5, 1 h, 2 h, 3 h, 4 h, 5 h, 6 h, 7 h, 8 h and 9 h using HPLC-analysis and the results compared to the control sample.

Determination of Tetrazine-Stability Under Resin Cleavage Conditions.

Trifluoroacetic acid (950 μL) and deionized water (25 μL) were added to a solution of of H-τ-OH 3 (5 mM, 20 μL, DMSO) and coumarin (5 mM, 5 μL, DMSO) and the resulting mixture exposed to sequential microwave irradiation intervals (0-11×2.5 min, 50 W). A control sample consisted of dimethyl sulfoxide (975 μL), which was added to a solution of of H-τ-OH 3 (5 mM, 20 μL, DMSO) and coumarin (5 mM, 5 μL, DMSO). At the end of each microwave irradiation interval, a sample was drawn, the amount of H-τ-OH 3 relative to coumarin was measured using HPLC-analysis and the results compared to the control sample.

### Additional Examples

We will be using the given invention for making a library of alpha-MSH peptides and conjugate these peptides to 4-5 different chelators. Purification will also work via the Tz/TCO (by running the crude materials over a resin, as shown by us before (Reiner T et al. (2011) Angew Chem Int Ed Engl 50, 1922-1925. [26]) This will allow us to generate libraries of imaging agents quickly, cheap and without a lot of effort, allowing large screenings of radioactive compounds, which will then translate to lead radiopharmaceuticals faster.

In one embodiment the invention relates to designing peptides which bind to cell surfaces without being internalized. They will then be injected with a complementary tracer and clicked in vivo. This is particularly useful if toxic compounds are being delivered, which can cause serious side-effects if they circulate longer.

This technique will be especially useful for ¹⁸F-imaging, since labeling of peptides with fluorine always represents a significant problem. Any peptide above 5 amino acids will significantly benefit from direct incorporation of Tetrazines.

Thus, specific compositions and methods of tetrazines/trans-cyclooctenes in solid phase synthesis of labeled peptides have been disclosed. It should be apparent, however, to those skilled in the art that many more modifications besides those already described are possible without departing from the inventive concepts herein. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

### Other Labels or Tracers

In one embodiment, the invention relates to designing peptides which bind to cell surfaces without being internalized. They will then be injected with a complementary tracer (which can be anything from the list below) and clicked in vivo. This is particularly useful if toxic compounds are being delivered, which can cause serious side-effects if they circulate longer.

Other than radiolabels, other imaging labels or tracers paired with peptides could include:

a) Fluorophores (e.g. Cy5, Cy5.5, Cy7, Alexa Fluor dyes, BODIPY dyes, Coumarin dyes, Fluorescin dyes)

b) Chromophores, including auxochromes and halochromes (e.g. beta-carotin, phenolphthalein, crystal violet, Orange G, Victoria Blue, Congo Red)

c) FRET pairs (e.g. Cy2-Cy3, Alexa Fluor 647-Alexa Fluor 750, CFP-YFP, GFP-mRFP, FITC-TRITC)

d) MRI imaging agents (e.g. Omniscan, Gd³⁺ binding chelators, Iron Oxide chelators, small molecules or nanoparticles)

e) CT contrast agents (e.g. Iohexol, Iopromide, Diatrizoate)

f) SPECT imaging agents (e.g. ¹²³I-MIBG, ¹³¹I-MIBG, ^{99m}Tc-HMPAO, ^{99m}Tc-tetrofosmin)

g) Cytotoxic materials (including alkylating agents and anti-metabolites, plant alkaloids and terpenoids, either Organometallic or organic or inorganic). (e.g. cis-Pt, carbo-Pt, taxol, cyclophosphamide, decetaxel)

h) Cherenkov-active isotopes (e.g. ¹⁸F, ⁶⁴Cu, ⁶⁸Ga)

i) Phosphorescent probes (e.g. containing ZnS, CaS, SrAl₂O₄, Ln silicates)

j) Luminescent probes (e.g. chemiluminescence, bioluminescence, electochemiluminescence, electroluminescence, crystallomuminescence, electrochemiluminescence, photoluminescence, radioluminescence, sonoluminescence, thermoluminescence)

k) Probes which represent a key component for the generation of Luminescence (e.g. luciferin, luciferase, ATP)

l) Photodynamic therapeutics and diagnostics (e.g. photosensitizers, photocleavable groups)

m) Nanoparticles (e.g. dextran-based nanoparticles, crosslinked iron oxide nanoparticles, silica based nanoparticles)

n) Quantum dots (e.g. Cadmium and Cadmium-Free Quantum dots, 1-1000 nm in size)

o) Redox-active organic or organometallic complexes (e.g. Ferrocene, Ferrocenium, cobaltocene, cobaltocenium, other metals or transition metal complexes)

p) Heat-sensitive materials (e.g. fluorite, feldspars, quartz)

q) Cold-sensitive materials (e.g. Bis(diethylammonium)tetrachlorocuprate, Leuco dye, spirolactones, fluorans, spiropyrans,)

r) Sensors for Reactive Oxygen species (ROS) (e.g trans-1-(2′-Methoxyvinyl)pyrene, 2-hydroxy-5-(triphenylphosphonium)hexylethidium, Amplex Red, 3′-(p-hydroxyphenyl) fluorescein)

s) Enzymes, peptides or other biomolecules (fluorescent or biologically active) (e.g. GFP, RFP, Cytochrome P50, HSP90, Somatostatin, Neurotensin Y, Substance P)

t) Small molecules markers (e.g. AZD2281, Taxol, Aspirin, ADP, ATP, NADP, Staurosporine, Estrogen)

u) Lanthanides (e.g. Lanthan, Cerium, Praesodymium, Neudymium, Promethium, Samarium, Europium, Gadolinium, Terbium, Dysprosium, Holmium, Erbium, Thumium, Ytterbium, Lutetium)

v) Actinides (e.g. Actinium, Thorium, Protactinium, Neptunium, Einsteinium)

w) Isotopically enriched non-radioactive isotopes (lanthanides, transition metals, metals). (e.g. ¹⁴²Nd, ¹⁴⁸Nd, ¹⁶⁶Er, ¹⁶⁸Yb, ¹⁷⁶Yb, ¹³⁹La,).

### First-in-Human Clinical Trial of a Click Chemistry-Based Strategy for Pet Imaging of Metastatic Colorectal Cancer

It is not intended that the present invention be limited by the nature of the label, which can be a radiolabel, fluorophore, microparticle, magnetic particle, charged particle, heavy metal or the like. Where a radiolabel is used, It is not intended that the present invention be limited by the radiolabel or the nature of the chemistry needed to complex the radiolabel. A variety of radiolabels are contemplated, including radioactive Cu, Zn, Ga as well as other radioactive metals and metalloids. With regard to the chemistry need to complex the radiolabel, NOTA chemistry has been exemplified for radioactive Cu. However, other chemistries are contemplated. For example, currently, 68Ga-labeled 1,4,7,10-tetraazacyclododecane-tetraacetic acid (DOTA)-complexes are the most widely used class of 68Ga radiotracers for PET, although DOTA is not optimal for 68Ga complexation. More recently, 1,4,7-triazacyclononane-triacetic acid (NOTA) and particularly triazacyclononane-phosphinate (TRAP) chelators have been shown to possess superior 68Ga binding ability. Thus, the present invention contemplates a variety of chemistries, including but not limited to DOTA, NOTA and TRAP chemistries, with a variety of radiolabels.

The remarkable specificity and affinity of antibodies make them extremely attractive vectors for the delivery of diagnostic and therapeutic radioisotopes to cancer cells [1]. Over the last two decades, a wide variety of antibody-based radiopharmaceuticals have been developed, employing isotopes ranging from ⁶⁴Cu for imaging to ²²⁵Ac for therapy Importantly, the relatively slow pharmacokinetics of antibodies require that the radioactive half-life of the isotope is compatible with the biological half-life of target-tissue localization of the vector.² In practice, this means that antibodies are often labeled with isotopes with multi-day physical half-lives (e.g. ¹²⁴I, ⁸⁹Zr, etc.). Such long physical half-lives allow time for the antibodies to accumulate in the tumor while simultaneously preventing the premature decay of their radioactive payload. However, this combination of long biological and physical half-lives gives rise to a critical complication: high radiation doses to non-target organs.

In order to circumvent these limitations, considerable attention has been dedicated to the development of methodologies that combine the advantages of antibodies with the pharmacokinetics of smaller molecules. One particularly appealing method relies on the decoupling of the targeting vector and the radioactivity and, as such, is called pre-targeting [3-9]. Generally, pre-targeting methodologies employ four steps: (1) the administration of an antibody with the ability to bind both an antigen and a radioligand; (2) the slow accumulation of the antibody in the tumor and its concomitant clearance from the blood; (3) the administration of a small molecule radioligand; and (4) the binding of the radioligand to the antibody followed by the rapid clearance of excess radioactivity. In essence, pre-targeting strategies allow for the exploitation of the exceptional affinity and specificity of antibody-based targeting vectors while simultaneously taking advantage of the rapid pharmacokinetics of small molecule-based radioligands.

Two pre-targeted imaging methodologies predominate in the literature: (1) those based on bispecific antibodies capable of binding both an antigen and a radiolabeled hapten and (2) those in which streptavidin modified antibodies and biotin-based radioligands are used [4, 6, 7, 10-16]. Both strategies are creative solutions and have proven promising in preclinical studies. Each, however, possesses significant limitations that constrain their long-term clinical applicability. For the former, these lie principally in the complexity and expense associated with the generation of bispecific antibodies. For the latter, the primary drawbacks have been the immunogenicity of the streptavidin-modified antibodies [10].

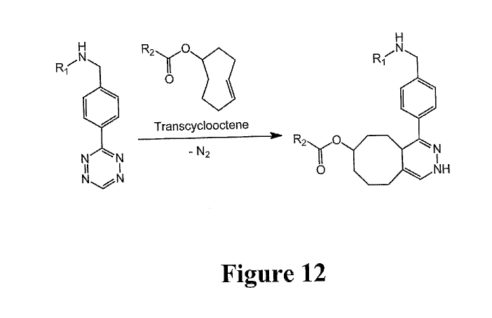

In recent years, a chemical methodology has been developed that has the potential to aid in the development of novel, antibody-based pre-targeting systems without these limitations: the rapid, selective, and bioorthogonal inverse electron demand [4+2] Diels-Alder (IEDDA) ‘click chemistry’ cycloaddition between a tetrazine (Tz) and a transcyclooctene (TCO) dienophile (FIG. 12) [11-15]. This ligation is high yielding, clean, exceptionally rapid (k1>30,000 M⁻¹s⁻¹), selective, and critically bioorthogonal. Indeed, the bioorthogonal nature of this ligation has been well-established both in vitro and in vivo. Neither of the two Diels-Alder components will react with any other biological functional group, and the reaction between the two components is unfazed by media, serum, and blood [11, 13-15]. To date, the ligation has been used in a variety of settings, including traditional and pre-targeted nuclear imaging with both ¹⁸F- and radiometal-based probes [15-20].

Results presented herein demonstrate the development and validation of an in vivo pre-targeted methodology based on the IEDDA reaction for the PET imaging of colorectal cancer [21], including the first-in-human clinical trial to assess the safety and efficacy of this methodology for imaging patients with metastatic colorectal cancer. Previous attempted to translate pre-targeted imaging modalities to the clinic in the past with mixed results at best [3, 4, 7]. However, unlike previous pre-targeting systems that relied on complex, genetically-engineered constructs, the click chemistry-based strategy disclosed herein is non-immunogenic, inexpensive, chemically simple, and poses no threat for inadvertent antibody-antigen cross-linking [21, 22].

Preclinical results demonstrate that in humans this methodology is able to produce PET images with quality and contrast comparable to directly radiolabeled antibodies but at only a small fraction of the radiation dose to the patient. The clinical trials outlined below represent just the third study to involve a radiopharmaceutical synthesized using click chemistry and the very first study in which click chemistry occurs within the human body. In addition, the clinical translation of this pre-targeted PET imaging strategy lends itself to clinical application of similar methods for radioimmunotherapy; a discipline which would also benefit from the reduction in background radiation dose offered by pre-targeted methodologies. This first-in-human clinical trial will have a significant impact on the health of patients. In the long term this work will have a transformational effect on the way antibodies are employed in diagnostic and therapeutic nuclear medicine.

Results presented herein outline the development and validation of a novel strategy for the pre-targeted ⁶⁴Cu-PET imaging of colorectal cancer based on the rapid and bioorthogonal inverse electron demand DielsAlder click reaction. Preclinical murine models of colorectal cancer have demonstrated that this methodology is capable of effectively delineating tumor tissue with high contrast, producing images with tumor-to-background activity ratios comparable to traditional, directly labeled antibodies. In addition, preclinical dosimetry studies revealed that this pre-targeted imaging methodology produces only a fraction of the background radiation dose to healthy tissue compared to directly labeled antibodies, making it a safer alternative to traditional immunoPET. Given these results, one embodiment the present invention contemplates an exploratory, first-in-human clinical trial (eIND) of this pretargeted PET imaging methodology in patients with metastatic colorectal cancer. Overall, this eIND study provides imaging and fundamental biodistribution, metabolism, and safety information. Two additional points of investigation include (1) assessing the ability of this pre-targeted imaging methodology to effectively delineate metastatic lesions in the liver with high tumor-to-background contrast and (2) determining the interval time between the injection of the antibody construct and the tetrazine radioligand that produces the optimal image contrast and tumor-to-background activity ratios.

Preliminary Results

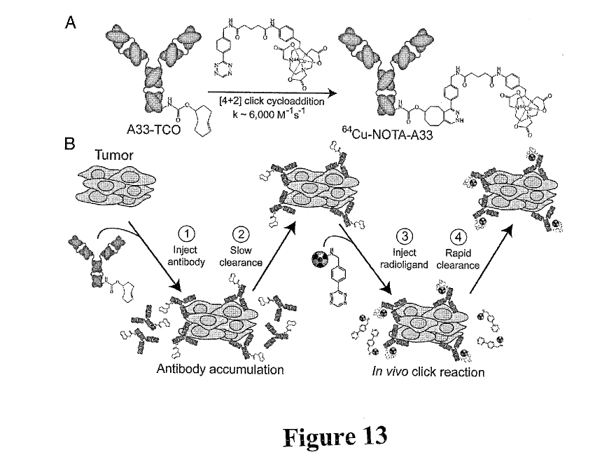

The inventors have developed a pre-targeted PET imaging methodology based on the inverse electron demand [4+2] cycloaddition reaction between tetrazine and transcyclooctene [65]. The system is comprised of four steps: (1) the injection of a mAb-TCO conjugate; (2) a localization time period during which the antibody accumulates in the tumor and clears from the blood; (3) the injection of the radiolabeled tetrazine; and (4) the in vivo click ligation of the two components followed by the clearance of excess radioligand (FIG. 13).

The first step in the creation of the pre-targeting system was the selection of a suitable antibody candidate. The huA33 antibody-antigen system was ultimately chosen since it is arguably one of the best studied antibodies from a human biodistribution point of view [23-27]. The huA33 antibody targets the A33 antigen, a transmembrane protein that is homologous to tight-junction associated proteins and is expressed in more than 95% of colon cancers and 50% of gastric cancers but in very few other tissues [27] Importantly, the huA33 antibody has been shown to exhibit surface persistence even when bound to the A33 antigen. This is an important trait in the context of pre-targeting, for the internalization and consequent sequestration of the antibody prior to the administration of the radioligand would severely reduce the likelihood of successful in vivo click ligations.

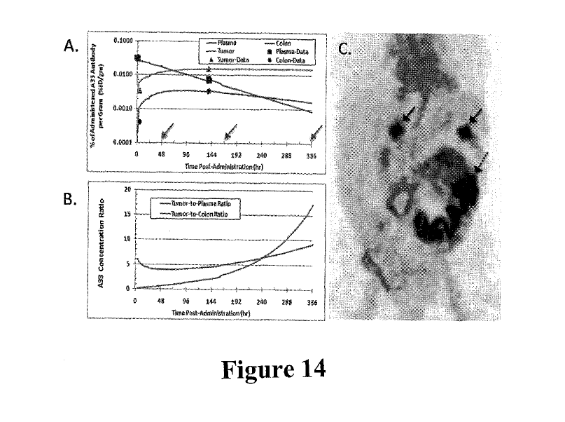

The humanized A33 antibody has been used extensively in animals and man, including for quantitative PET imaging. Indeed, a previous study of 25 patients with colorectal cancer metastatic to the liver, 13 of which had synchronous primary tumors [26], the inventors identified excellent tumor targeting and were able to show that the uptake of the radioimmunoconjugate was driven by antigen expression. As part of this trial, the inventors collected extensive immunokinetic data using a combination of quantitative imaging, blood and tissue collection, and whole body probe counts. FIG. 14A shows the quantitative plasma, tumor, and colon clearance curves obtained after injection of 10 mCi of ¹²⁴I-huA33 into a patient with colon cancer metastatic to the liver. A representative PET image obtained at 5 days after injection shows uptake in tumors in liver and splenic flexure (FIG. 14C). These images along with graphs of tumor-to-background ratios (FIG. 14B) reveal tumor-to-colon and tumor-to-plasma ratios sufficient for imaging metastatic lesions and primary tumors.

Despite the positive results with ¹²⁴I-huA33, the use of a pre-targeted strategy with huA33 provides significant improvements. Kinetic models have shown that there is a differential retention of antibody on tumor which is significantly greater than retention of antibody in normal tissues including blood and colon, two relatively radiosensitive tissues [23, 24, 26]. Pre-targeting is an ideal way to exploit this retention difference to improve tumor-to-background contrast. In one embodiment the tetrazine radioligand is injected after the shedding of the antibody from the normal colon but before the departure of the antibody from the cancerous tissue. For example, if the radioligand were injected 2 weeks (288 h) after the primary injection of huA33-TCO, the tumor-to-colon and tumor-to-blood ratios of the antibody will be more than 10-to-1 and 20-to-1, respectively. A pre-targeted strategy will also provide benefits with regard to dosimetry. For example, in the aforementioned clinical trial, the whole body dose of ¹²⁴I-huA33 was estimated to be around 0.45 mGy/MBq. Based on the preclinical data, the whole body absorbed dose for the ⁶⁴Cu-pretargeting approach will be 0.008 mGy/MBq.

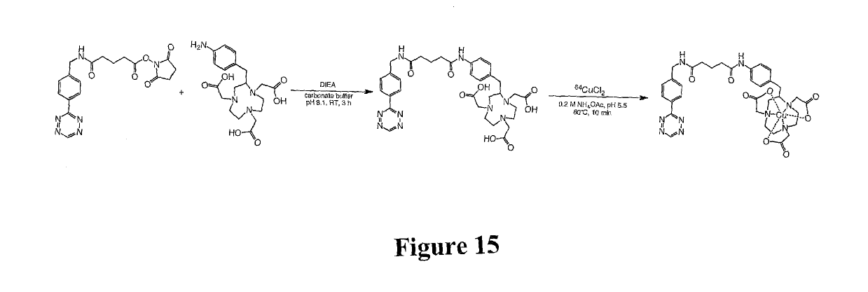

The next step is the synthesis and characterization of the molecular components. ⁶⁴Cu was chosen due to its advantageous imaging properties and intermediate half-life (12.7 h), and NOTA was selected as the chelator due to its high in vivo kinetic and thermodynamic stability with ⁶⁴Cu²⁺. A NOTA-modified tetrazine (TzNOTA) was synthesized, characterized, and radiolabeled with ⁶⁴Cu to yield the purified radioligand in 87±3% decay-corrected yield, >99% radiochemical purity, and a specific activity of 8.9±1.2 MBq/μg (FIG. 15). TCO-modified huA33 (huA33-TCO) was constructed via reaction of the antibody with an NHS ester of TCO. Subsequent characterization revealed that the antibody conjugates bore 4.2±0.6 TCO/mAb and maintained an immunoreactivity of >95%.

Following the synthesis and characterization of the components of the system, in vivo pre-targeted biodistribution and PET imaging experiments were performed. For the pre-targeted biodistribution experiments, mice bearing SW1222 xenografts were administered huA33-TCO (100 μg) via tail vein injection. After a 24 h interval, the mice were administered ⁶⁴Cu-Tz-NOTA via tail vein injection. The data reveal rapid accumulation and retention of activity in the tumor, with 4.1±0.3% ID/g at 1 h p.i and 4.2±0.3% ID/g at 12 h p.i. (Table 1). The compound is excreted through the feces, with high levels of activity at 1 h p.i. dropping to 0.9±0.1% ID/g at 12 h, and the amount of uptake in other tissues remains very low, generally less than 0.3% ID/g. Further, the rapid and unchanging uptake in the tumor indicates that ⁶⁴Cu-Tz-NOTA is clicking with huA33-TCO at the tumor, rather than clicking with huA33-TCO in the blood that then travels to the tumor.

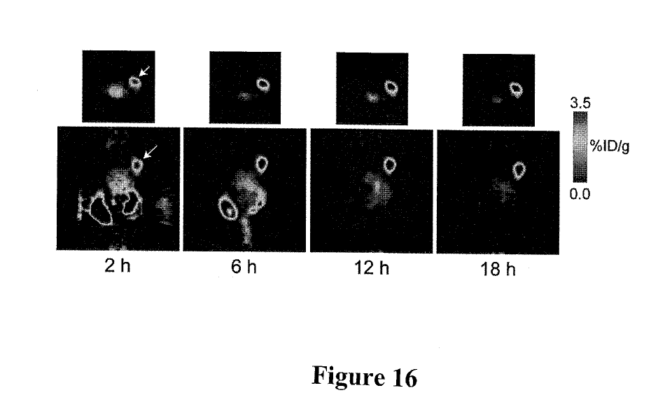

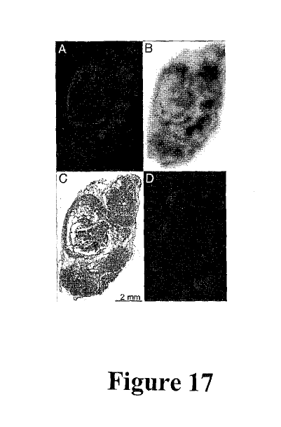

PET imaging experiments were performed in a similar manner, the only change being higher amounts of injected activity. Tumor uptake is apparent at 2 h post-injection, though, consistent with the biodistribution, it is much less pronounced than the uptake in the gut (feces) at this time point. Over the course of the experiment, however, the fecal radioactivity clears, resulting in excellent delineation of tumor from background at 12 and 18 h post-injection, in agreement with the 26.6±6.6 tumor: muscle activity ratio measured at 12 h in the biodistribution study (FIG. 16). Imaging experiments in which 300 μg of huA33-TCO were administered showed similar results; however, these experiments revealed higher activity levels in the blood, suggesting a greater incidence of the in vivo click reaction with huA33-TCO in the blood. Control imaging experiments reinforced that the in vivo click reaction is responsible for the accumulation of radioactivity in the tumor: controls employing ⁶⁴Cu-Tz-NOTA alone, unmodified huA33 rather than huA33-TCO, and a vast excess of unlabeled tetrazine all resulted in minimal uptake of activity in the tumor. In addition, pre-targeting with a nonspecific IgG-TCO construct resulted in apparent yet slight tumor uptake (<1.5% ID/g), which is to be expected given that non-targeted antibodies often accumulate in tumors due to malformed vasculature. Further, experiments in which tumors were resected after in vivo pre-targeting with a near infrared-fluorophore-labeled huA33-TCO and ⁶⁴Cu-Tz-NOTA produced histological and autoradiographical data that further illustrated the remarkable selectivity of the in vivo click ligation: the microscopic co-localization between the ⁶⁴Cu-labeled tetrazine and the fluorophore- and TCO-labeled antibody is nearly perfect (FIG. 17).

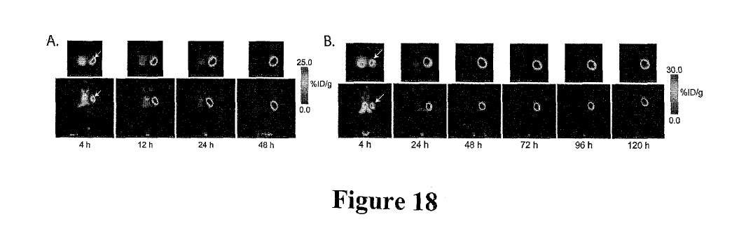

As a basis for comparison, biodistribution and imaging experiments were also performed with huA33 directly labeled with either ⁶⁴Cu-NOTA or ⁸⁹Zr-DFO (FIG. 18). These experiments revealed that higher absolute tumor uptake values were observed with the directly labeled antibodies: for example, maxima of 33.1±7.9% ID/g at 24 h for ⁶⁴Cu-NOTA-huA33 and 43.3±9.0% ID/g at 24 h for ⁸⁹Zr-DFO-huA33. However, the far lower non-target tissue uptake values created by the pre-targeting system resulted in equivalent if not superior tumor-to-background ratios (e.g., tumor-to-muscle ratios at 24 h: 27.0±7.4 for pre-targeted ⁶⁴Cu, 29.8±6.7 for ⁶⁴Cu-NOTA-huA33, and 12.7±3.3 for ⁸⁹Zr-DFO-huA33) and PET images of comparable quality.

Using the biodistribution data, dosimetry calculations were performed for the pre-targeting system and the two directly labeled antibodies in order to determine the background radiation dose associated with each. The data clearly indicate that the pre-targeting system provides a significant dosimetric advantage compared to the ⁸⁹Zr-labeled antibody: the effective dose with ⁶⁴Cu pre-targeting is 0.012 mSv/mBq, while that due to [⁸⁹Zr]-DFO-huA33 is 0.41 mSv/mBq (Table 2). Importantly, an even more pronounced disparity exists when considering the mean absorbed dose to the bone, specifically red marrow and osteogenic cells; with [⁸⁹Zr]-DFO-huA33, the dose to these tissues is 0.84 and 1.64 mGy/mBq, respectively, compared to 0.014 and 0.023 mGy/mBq with ⁶⁴Cu pre-targeting. Not surprisingly, given the shorter half-life of ⁶⁴Cu, these advantages are lessened when comparing the pre-targeting system to ⁶⁴Cu-NOTA-huA33, though an overall dosimetric advantage still exists for the pre-targeting system, particularly with regard to bone marrow.

By applying this pre-targeted system for PET imaging based on the in vivo bioorthogonal ‘click’ reaction between tetrazine and transcyclooctene to a model system the inventors found that this methodology produces high quality images with tumor-to-background contrast comparable to that achieved with directly radiolabeled antibodies. Further, the system produces these images at a dramatically reduced absorbed dose to non-target organs compared to imaging with antibodies directly labeled with long-lived isotopes.

Experimental Methods

The present eIND study is designed to establish imaging and fundamental biodistribution, metabolism, and safety information. Patients selected for removal of colorectal metastases are injected with the huA33-TCO conjugate. Subsequently, the ⁶⁴Cu-Tz-NOTA radioligand is injected 2 days, 1 week, or 2 weeks later, and the images are compared in cohorts of 5 patients each (proposed times of injection are illustrated by the red arrows on the clearance curve in FIG. 14A). PET imaging occurs at 4 time points after the injection of ⁶⁴Cu-Tz-NOTA: 1, 24, 48, and 72 hours.

Toxicology/Pharmacology and IND Preparation.

Studies are conducted in accordance with recommendations from U.S. Department of

Health and Human Services with the goal of providing data for an Investigational New Drug (IND) application. Given their substantial experience with the clinical translation of radiopharmaceuticals, the inventors not anticipate major issues in the translation of ⁶⁴Cu-Tz-NOTA.

Toxicity/pharmacology studies are conducted by the SKI Antitumor Assessment Core Facility at MSKCC. Microdose studies are performed in male mice and are designed to (1) identify a safe starting dose for humans, (2) evaluate organs that may be the targets of toxicity, (3) estimate the margin of safety between a clinical and a toxic dose, and (4) predict pharmacokinetic and pharmacodynamic parameters. A large multiple (e.g., 100×) of the proposed human dose will be examined. Scaling from animals to humans based on body surface area is used to select the dose for use in clinical trials. Scaling is based on pharmacokinetic/pharmacodynamic modeling. Normal-organ radiation absorbed doses and the effective dose for ⁶⁴Cu-Tz-NOTA in humans have already been estimated based on preclinical studies in murine models of colorectal cancer [21].

Based on the proven safety record of both unmodified huA33 and ¹²⁴I-huA33 in humans, the inventors will apply to the IRB for an exemption from toxicology screening for the huA33-TCO conjugate[24, 26]. The inventors are confident that the covalent attachment of 2-3 biologically-inert, ˜200 Da moieties to an antibody that weights >150,000 Da will not alter its pharmacological profile. Therefore, the toxicology and pharmacology studies will be performed solely on the Cu-Tz-NOTA construct.

Preparation of huA33-TCO

The conjugated huA33-TCO construct is prepared in sterile and pyrogenfree form under cGMP conditions. cGMP preparation presents limited issues, as MSKCC already has a cGMP manufacturing facility in place.

GMP Production

Production of huA33-TCO and the precursor Tz-NOTA is performed through the MSKCC Radiochemistry and Molecular Imaging Probe Core Facility, which has successfully produced constructs (e.g., DFO-J591) currently under clinical study under an FDA eIND.

CMC Section

The Cyclotron Core at MSKCC is responsible for running three full validation runs with full QC for generating the CMC section for an IND, and will generate standard operating procedures (SOPs) and maintain batch records. This process has been successful for over >10 small molecules to date.

Imaging Studies

A clinical protocol (under the auspices on the FDA eIND) is currently being developed to study a pilot group of 15 patients. The inclusion criteria for this study include: (i) metastatic colorectal carcinoma, histologically confirmed at MSKCC; (ii) candidates for clinically indicated surgery/biopsy; (iii) expected survival of >3 months; (iv) Karnofsky performance status ≧70 (ECOG 0 or 1); (v) the following laboratory results within the last 2 weeks prior to study day 1 [absolute neutrophil count (ANC) ≧1.5×10⁹/L; platelet count ≧75×10⁹/L; serum bilirubin ≧2.5×mg/dL; serum creatine ≧2.0×mg/dL; white blood count ≧3000 mm³]; (vi) age ≧18 years; and (vii) signing of a study-specific informed consent prior to study entry and sign the IRB-approved consent form. The exclusion criteria for this study include: (i) clinically significant cardiac disease; (ii) active CNS tumor involvement; (iii) previous treatment with huA33 or its fragment and/or a positive test for huA33 HAHA; (iv) lack of availability for immunological and clinical follow-up assessments; (v) participation in any other clinical trial involving another investigational agent with 4 week prior to enrollment; (vi) women who are pregnant or breast-feeding. Patients will be imaged using the dedicated research PET/CT scanner (GE Discovery STE, USA) at MSKCC.

Pre-targeted PET Imaging with huA33-TCO and ⁶⁴Cu-Tz-NOTA:

Both huA33-TCO and ⁶⁴Cu-Tz-NOTA will be prepared in sterile and pyrogen free form under cGMP conditions. On study day 1, patients are administered a single intravenous infusion of 10 mg of huA33-TCO. 2 days (n=5), 7 days (n=5), or 14 days (n=5) after the initial injection of huA33-TCO, the patients are injected with ˜185 MBq (5 mCi) of ⁶⁴Cu-Tz-NOTA. The amounts of radioactivity selected for administration to each subject in the study are in accordance with the radiation protection principle “as low as reasonably achievable” (ALARA), while ensuring image quality that is suitable for the planned analyses. The MIRD dosimetry program, OLINDA, (Vanderbilt University) will be used for dosimetry calculation.

A 30-minute dynamic PET emission scan is initiated coincident with the radiotracer injection. The radiotracer profile obtained from the dynamic imaging data along with plasma time activity profile analysis is used to investigate compartment models aimed at determining the pharmacokinetics of the tracer. Datasets are used to determine time-activity curves for ⁶⁴Cu-Tz-NOTA in blood, tumor, and organs/tissues of interest. After the dynamic scan, patients undergo a whole body scan (3-minutes per bed position). The combined duration of the dynamic and whole body ⁶⁴Cu-Tz-NOTA scan is approximately 1 h, after which the patient dismounts from the table. Patients then return to the PET/CT scanner for whole body static scans at 24, 48, and 72 h p.i. for whole body static scans. Venous blood samples are collected at each of the PET studies in order to determine the clearance of ⁶⁴Cu-Tz-NOTA from the blood and to detect potential metabolites by HPLC. Toxicity and safety data will be reviewed on an ongoing basis after the injection of huA33-TCO and ⁶⁴Cu-Tz-NOTA, which will include pre-administration events, physical examination, injection-site monitoring, vital signs (systolic and diastolic BP, heart rate, body temperature, and respiration rate), 12-lead ECG, clinical laboratory variables (serum biochemistry and hematology), and adverse events. Patients are encouraged to report any symptoms after injection of huA33-TCO and ⁶⁴Cu-Tz-NOTA.

Radiation doses are calculated from the PET imaging data and time activity curves as described. Secondary endpoints include the visualization of primary tumors and metastatic lesions by ⁶⁴Cu-Tz-NOTA and assessment of the tumor-to-background specifically; the tumor-to-liver and tumor-to-colon activity ratios for the tracer. To this end, reconstructed PET/CT images are displayed on an AW Suite workstation and reoriented into maximum intensity projection (MIP), transaxial, coronal, and sagittal images. The PET images are interpreted qualitatively and semi-quantitatively on a lesion by lesion basis. Semi-quantitative analysis will be employed as follows: (a) regions of interest (ROI) is placed around lesions in order to obtain standardized uptake value (SUV) parameters; (b) SUV data are recorded along with volumetric and positional information in a standardized form; (c) ROI's will also be placed in selected organs to measure the organ and background activity concentration and its changes over time; and (d) profiles will be generated in units of SUV versus time in order to determine optimal time points for tumor imaging.

All SUV measurements are summarized using mean, median, range, and counts where appropriate and a repeated measures analysis of variance model will be used to relate the SUVs to the tissue regions. The F-statistic for the overall model as well as pairwise comparisons of the SUVs for each tissue type are investigated. In addition, descriptive statistics for the SUVs are done on a subject basis and a per lesion basis. SUVs representing uptake and retention of ⁶⁴Cu-Tz-NOTA into non-malignant colon or liver pathology and normal colon or liver tissue are also be measured, and graphs of the SW distributions are prepared by tissue type. Uptake and retention may not be observed in tumors which are smaller than the spatial resolution of PET imaging. Therefore, the primary analysis are done for all tumors and separately for tumors >6 mm.

If the initial analysis of the first 5 patients in this study reveals an unexpectedly high dose rate to any particular organ, dose reductions in the remaining patients will be made. Conversely, if preliminary results indicate that normal-organ absorbed dose limits will not be exceeded, then the administered activity of ⁶⁴Cu-Tz-NOTA will be increased to improve image quality. It is possible that the liver and bowel uptake due to catabolism of the radiotracer will interfere with image contrast of the metastases. The later imaging time points chosen (i.e. 48 h and 72 h) should permit enough time for clearance; however, if the initial analyses reveal that these time points are not sufficient to allow for clearance, additional time points will be added.

### Materials and Methods

Reagents and General Procedures

All chemicals, unless otherwise noted, were acquired from Sigma-Aldrich and used as received without further purification. All water used was ultra-pure (>18.2 MΩcm²¹), and all dimethyl sulfoxide was of molecular biology grade (>99.9%). Both 2,2′,2″-(2-(4-aminobenzyl)-1,4,7-triazonane-1,4,7-triyl)triacetic acid (NH₂-Bn-NOTA) and p-isothiocyanatobenzyl-desferrioxamine (SCN-DFO) were purchased from Macrocyclics, Inc. Amine-reactive transcyclooctene (TCO-NHS) and amine-reactive tetrazine (Tz-NHS) were synthesized as described previously (13). Humanized A33 antibody was generously provided by the Ludwig Institute for Cancer Immunotherapy.

⁶⁴Cu was purchased from Washington University, St. Louis, where it was produced on the Washington University School of Medicine Cyclotron (model CS-15; Cyclotron Corp.) by the ⁶⁴Ni(p,n)⁶⁴Cu reaction and purified to yield 64Cu—CuCl₂ with a specific activity of 7.4-14.8 GBq/μg. ⁸⁹Zr was produced at Memorial Sloan-Kettering Cancer Center on a TR19/9 cyclotron (Ebco Industries Inc.) via the ⁸⁹Y(p,n)⁸⁹Zr reaction and purified to yield ⁸⁹Zr with a specific activity of 196-496 MBq/μg. Activity measurements were made using a CRC-15R Dose Calibrator (Capintec). For the quantification of activities, experimental samples were counted on an Automatic Wizard (2) γ-Counter (Perkin Elmer). The labeling of antibodies with both ⁶⁴Cu and ⁸⁹Zr was monitored using silica gel-impregnated instant thin-layer chromatography (TLC) paper (Pall Corp.) and analyzed on an AR-2000 radio-TLC plate reader (Bioscan Inc.). The human colorectal cancer cell line SW1222 was obtained from the Ludwig Institute for Cancer Immunotherapy and grown by serial passage. All in vivo experiments were performed according to protocols approved by the Memorial Sloan-Kettering Institutional Animal Care and Use Committee.

Modification of A33 with TCO-NHS

A33 (2 mg) was dissolved in 500 μL of phosphate-buffered saline (PBS, pH 7.4), and the pH of the solution was adjusted to 8.8-9.0 with NaHCO₃ (0.1 M). To this solution was added an appropriate volume of TCO-NHS in N,N-dimethylformamide (10 mg/mL) to yield a TCO: mAb reaction stoichiometry of 10:1. The resulting solution was incubated with gentle shaking for 30 min at room temperature. After 30 min, the modified antibody was purified using centrifugal filter units with a 50,000-Dalton molecular weight cutoff (Amicon Ultra 4; Millipore Corp.) and PBS.

Radiolabeling of Tz-Bn-NOTA with ⁶⁴Cu

A solution of Tz-Bn-NOTA (5-6 pg) in NH₄OAc buffer (0.2 M, pH 5.5) was prepared. ⁶⁴CuCl2 in 0.1 M HCl (74-81 MBq) was added to this solution, and the reaction mixture was heated to 80° C. for 10 min. After heating, the solution was allowed to cool to room temperature and purified via high-performance liquid chromatography (HPLC) (retention time, 9.6 min). Solvent was removed via rotary evaporation to yield purified product in an 87%±3% decay-corrected yield with a radiochemical purity of more than 99% and a specific activity of 8.9±1.2 MBq/μg.

Pre-Targeted PET Imaging

Pre-targeted PET imaging experiments were conducted on a micro-PET Focus 120 scanner (Concorde Microsystems). Mice bearing subcutaneous SW1222 xenografts (100-150 mm³, 9-12 days after inoculation) were administered A33-TCO (100 μg in 200 μL of 0.9% sterile saline) via tail vein injection. After 24 h, mice were administered ⁶⁴Cu-Tz-Bn-NOTA (10.2-12.0 MBq [275-325 μCi] in 200 μL of 0.9% sterile saline) via tail vein injection (t=0). Approximately 5 min before PET imaging, mice were anesthetized by inhalation of a 2% isoflurane (Baxter Healthcare):oxygen gas mixture and placed on the scanner bed; anesthesia was maintained using a 1% isoflurane:gas mixture. PET data for each mouse were recorded in list mode at various time points between 2 and 18 h.

### Results

System Design

The first step in the development of the pre-targeting methodology was the design of the model system. Five components needed to be chosen: antibody, tetrazine, dienophile, radionuclide, and chelator. The antibody selected, A33, is a humanized antibody that targets the A33 antigen, a transmembrane glycoprotein present in more than 95% of human colorectal cancers [20]. In addition, because of its association with tight junction proteins, the A33 antigen has been shown to exhibit surface persistence, even when bound to the targeting antibody [21]. This is an extremely important trait in the context of pre-targeting: the internalization and consequent sequestration of the antibody before the administration of the radioligand would severely reduce the likelihood of in vivo click ligations. 3-(4-benzylamino)-1,2,4,5-tetrazine and transcyclooct4-en-1-yl hydrogen carbonate were chosen as the tetrazine-dienophile click pair because of their convenient conjugation handles and their balance of relative stability with rapid reaction kinetics (k 5 6,000^{M21s21} at 37° C.) [13,14]. Finally, ⁶⁴Cu was selected as the radioisotope. Despite the utility and ubiquity of ¹⁸F, a radiometal-based system offers greater versatility and modularity. In particular, ⁶⁴Cu displays favorable imaging characteristics and exhibits a half-life (12.7 h) normally considered suboptimal for clinical antibody imaging. The choice of ⁶⁴Cu, in turn, narrowed the choice of chelator, and NOTA was selected because of its relatively rapid chelation kinetics and high in vivo stability with ⁶⁴Cu [22].

Synthesis, Characterization, and Reactivity of Components

The NOTA-modified tetrazine (Tz-Bn-NOTA) was synthesized in high yield (0.95%) via peptide coupling from NH₂-Bn-NOTA and 3-(4-benzylamino)-1,2,4,5-tetrazine bearing an amine-reactive linker (FIG. 15). The compound was purified using reversed-phase C₁₈ HPLC and characterized by ultraviolet-visible spectroscopy, proton nuclear magnetic resonance spectroscopy, and electrospray ionization mass spectrometry. Tz-Bn-NOTA was, in turn, labeled with ⁶⁴Cu via incubation with ⁶⁴Cu—CuCl₂ at 90° C. for 10 min in NH₄OAc buffer. The radioligand was obtained in 87%±3% decay-corrected yield and purified via reversed-phase HPLC to more than 98% radiochemical purity, and its identity was confirmed against a cold Cu-Tz-Bn-NOTA standard. The specific activity of the purified ⁶⁴Cu-Tz-Bn-NOTA was 8.9±1.2 MBq/mg (n=10).

The stability of the radioligand was assayed in vitro by incubation in human serum at 37° C. and subsequent HPLC analysis. The ⁶⁴Cu-Tz-Bn-NOTA remained relatively stable after 1 h, with 93.0%±2.5% intact. With time, however, decomposition of the ⁶⁴Cu-Tz-Bn-NOTA becomes apparent: at 12 h, only 31.6%±6.1% of the radioligand remained intact. In addition, in vivo stability experiments were performed, and these produced similar results, although some acceleration in decomposition can be noted: 77%±3% of the ⁶⁴Cu-Tz-Bn-NOTA is intact in the blood after 60 min, followed by 47%±2% after 2 h and 22%±3% after 6 h. Although greater stability may be preferable, the speed of the cycloaddition reaction and the rapid pharmacokinetics of the radioligand led us to believe it unlikely that poor serum stability at later time points would limit the system.

The TCO-modified A33 (A33-TCO) was constructed via reaction of the antibody with 10 molar equivalents of the N-hydro-succinimidyl ester of TCO (TCO-NHS) for 30 min at room temperature in PBS adjusted to pH 8.8-9.0 with NaHCO₃ (0.1 M), and the bioconjugate was purified using centrifugal filtration. To determine the number of TCO per monoclonal antibody, the antibody was ligated with a 50-fold molar excess of Tz-Bn-NOTA in PBS (pH 7.4) and purified via size-exclusion chromatography. An isotopic dilution was performed on this NOTA-modified product to determine a degree of labeling of 5.0±0.9 NOTA/mAb, in turn suggesting a degree of labeling of 5 TCO/mAb, given the quantitative nature of the click ligation [18].

With the 2 system components in hand, the reaction between A33-TCO and ⁶⁴Cu-Tz-Bn-NOTA was next investigated. A33-TCO was incubated with ⁶⁴Cu-Tz-Bn-NOTA in 1:5, 1:1, or 5:1 Tz-to-mAb molar ratios in either PBS or serum at 37° C. for 30 min. As analyzed by radio-TLC, the reaction proceeded to near-complete conversion, with the 1:5 Tz-to-mAb reaction providing yields of 91.2%±0.9% and 89.6%±1.8% in PBS and serum, respectively, with only slightly diminished yields with the other ratios [18]. As negative controls, the reactions of free ⁶⁴Cu with A33-TCO and ⁶⁴Cu-Tz-Bn-NOTA with unmodified A33 were investigated, and in each case less than 1% association of the activity with the antibody was observed.

Finally, to probe the effect of the TCO conjugation on the immunoreactivity of the antibody, A33-TCO was reacted with a 5-fold excess of ⁶⁴Cu-Tz-Bn-NOTA in PBS and purified via size-exclusion chromatography to form ⁶⁴Cu-NOTA-A33 in more than 99% radiochemical purity and a specific activity of 125.8±11.1 MBq/mg. Immunoreactivity assays with A33 antigen-expressing SW1222 human colorectal cancer cells yielded an immunoreactivity for the bioconjugate of 95.6%±4.3%.

Pre-Targeted PET Imaging and Biodistribution

After the synthesis and characterization of the components of the system, the next step was to perform in vivo pre-targeted biodistribution and PET imaging experiments. Not surprisingly, one of the most important variables in the development of a pre-targeted system is the length of the interval between the injection of the antibody and the administration of the radioligand. In this case, biodistribution studies with clicked and preassembled ⁶⁴Cu-NOTA-A33 indicate that the maxi-mum loading of ⁶⁴Cu-NOTA-A33, and thus by proxy A33-TCO, in the tumor occurs at 24 h after injection, and relatively little antibody remains in the blood pool at the same time point. Thus, it becomes clear that 24-48 h represents a promising interval between the administration of antibody and the subsequent injection of radioligand.

For the pre-targeted biodistribution experiments, mice bearing subcutaneous SW1222 xenografts were administered A33-TCO (100 mg) via tail vein injection. After a 24-h interval for the accumulation of the antibody in the tumor and its simultaneous clearance from the blood, the mice were administered ⁶⁴Cu-Tz-Bn-NOTA (0.55-0.75 MBq, 0.06-0.09 mg, a 0.14-0.18 Tz-to-A33 ratio) via tail vein injection. The data reveal rapid accumulation and retention of radioactivity in the tumor, with 4.1±0.3 percent injected dose per gram (% ID/g) at 1 h after injection, 4.2±0.8% ID/g at 12 h, and 4.0±0.9% ID/g at 24 h (Table 1). The compound is excreted through the feces, with high levels of activity at 1 h after injection dropping to 2.5±0.1% ID/g at 12 h. The amount of uptake in all other tissues remains low, generally less than 1% ID/g. The rapid and steady uptake in the tumor indicates that 64Cu-Tz-Bn-NOTA is clicking with A33-TCO localized at the tumor, rather than clicking with A33-TCO in the blood pool that subsequently accumulates in the tumor.

PET imaging experiments were performed in a similar manner; the only change was the higher amount of injected activity (10.2-12.0 MBq, 1.2-1.4 mg, a 2.5-2.8 Tz-to-A33 ratio) (FIG. 16). Tumor uptake is apparent at 2 h after injection, although consistent with the biodistribution, it is much less than the uptake in the gut (feces). However, over the course of the experiment, the fecal radioactivity clears dramatically, resulting in excellent delineation of tumor from background by 12 h after injection, in agreement with the 26.6±6.6 tumor-to-muscle activity concentration ratio measured at the same time point in the biodistribution study.

Imaging experiments in which 300 mg of A33-TCO were administered showed similar results; however, these experiments revealed higher activity levels in the blood, suggesting a greater incidence of in vivo click reactions in the blood in addition to ligations at the tumor. Similarly, imaging experiments performed with a pre-targeting interval of 12 h resulted in a higher degree of activity in the blood, likely for the same reason. Control imaging experiments using ⁶⁴Cu-Tz-Bn-NOTA alone, unmodified A33 rather than A33-TCO, and a vast excess of unlabeled tetrazine resulted in minimal uptake of activity in the tumor, in all cases less than 0.25% ID/g. Further, pre-targeting with a nonspecific IgG-TCO construct resulted in apparent yet slight tumor uptake (<1.5% ID/g), not surprising given the tendency of non-targeted antibodies to accumulate in tumors because of the enhanced permeability and retention effect [23]. Finally, although preliminary pilot imaging experiments with longer pre-targeting intervals (e.g., 72 h) strongly suggest that the 24-h interval used strikes the optimal balance between tumor uptake and background clearance, further interval optimization studies are currently under way in our laboratory.

Although the PET imaging experiments form the core of this investigation, they only allow us to monitor the fate of 1 of the 2 components in the system: the radioligand. To circumvent this issue, A33-TCO was modified with the near-infrared fluorophore AlexaFluor680 (AF680) to yield A33-TCO-AF680 with a degree of labeling of 1.6 AF680/mAb. Tumor-bearing mice were then injected with A33-TCO-AF680 (100 mg), imaged with fluorescence at 24 h after antibody injection, injected immediately thereafter with ⁶⁴Cu-Tz-Bn-NOTA (10.2-12.0 MBq [275-325 mCi]), and then imaged with both fluorescence and PET 12 h after radiotracer injection. Not surprisingly, the PET imaging results from these experiments clearly mirror those reported above for the non-fluorescent antibody.

More important than the in vivo imaging experiments, this multimodality approach facilitated the tracking of the 2 system components at the microscopic level via autoradiography and fluorescence microscopy (FIG. 17). To this end, ex vivo analysis of resected tumors was performed to determine the relative distributions of A33-TCO-AF680 and ⁶⁴Cu-Tz-Bn-NOTA. A close correspondence of the 2 components was observed, with regions of high A33-TCO-AF680 uptake spatially matching regions of high ⁶⁴Cu-Tz-Bn-NOTA uptake. Further, A33-TCO-AF680 was associated exclusively with regions containing tumor cells and did not appear to associate with stromal or muscle tissue. More than any other piece of data in this investigation, the clear and striking microscopic co-localization of the fluorescently labeled A33-TCO and the ⁶⁴Cu-Tz-Bn-NOTA illustrates the occurrence of the in vivo click ligation. Finally, A33-TCO-AF680 shows a distinct cell surface distribution, which is easily visualized when compared with the nuclear counterstain 4′,6-diamidino-2-phenylindole and is consistent with the cell surface expression of the A33 antigen.

### Additional Embodiments

The specificity of antibodies have made immunoconjugates promising vectors for the delivery of radioisotopes to cancer cells; however, their long pharmacologic half-lives necessitate the use of radioisotopes with long physical half-lives, a combination that leads to high radiation doses to patients. Therefore, the development of targeting modalities that harness the advantages of antibodies without their pharmacokinetic limitations is desirable. To this end, we report the development of a methodology for pre-targeted PET imaging based on the bioorthogonal Diels-Alder click reaction between tetrazine and transcyclooctene. A proof-of-concept system based on the A33 antibody, SW1222 colorectal cancer cells, and ⁶⁴Cu was used. The huA33 antibody was covalently modified with transcyclooctene, and a NOTA-modified tetrazine was synthesized and radiolabeled with ⁶⁴Cu. Pre-targeted in vivo biodistribution and PET imaging experiments were performed with athymic nude mice bearing A33 antigen-expressing, SW1222 colorectal cancer xenografts. The huA33 antibody was modified with transcyclooctene to produce a conjugate with high immunoreactivity, and the ⁶⁴Cu-NOTA-labeled tetrazine ligand was synthesized with greater than 99% purity and a specific activity of 9-10 MBq/mg. For in vivo experiments, mice bearing SW1222 xenografts were injected with transcyclooctene-modified A33; after allowing 24 h for accumulation of the antibody in the tumor, the mice were injected with 64Cu-NOTA-labeled tetrazine for PET imaging and biodistribution experiments. At 12 h after injection, the retention of uptake in the tumor (4.1±0.3 percent injected dose per gram), coupled with the fecal excretion of excess radioligand, produced images with high tumor-to-background ratios. PET imaging and biodistribution experiments performed using A33 directly labeled with either 64Cu or 89Zr revealed that although absolute tumor uptake was higher with the directly radiolabeled antibodies, the pre-targeted system yielded comparable images and tumor-to-muscle ratios at 12 and 24 h after injection. Further, dosimetry calculations revealed that the 64Cu pretargeting system resulted in only a fraction of the absorbed background dose of A33 directly labeled with 89Zr (0.0124 mSv/MBq vs. 0.4162 mSv/MBq, respectively). Conclusion: The high quality of the images produced by this pretargeting approach, combined with the ability of the methodology to dramatically reduce non-target radiation doses to patients, marks this system as a strong candidate for clinical translation.

The remarkable specificity and affinity of antibodies make them extremely attractive vectors for the delivery of diagnostic and therapeutic radioisotopes to cancer cells (1). Over the last 2 decades, a wide variety of antibody-based radiopharmaceuticals has been developed using isotopes ranging from ⁶⁴Cu for imaging to 225Ac for therapy. Importantly, the relatively slow pharmacokinetics of antibodies requires that the radioactive half-life of the isotope be compatible with the biologic half-life of the target-tissue localization of the vector (2). In practice, this means that antibodies are often labeled with isotopes with multiday physical half-lives. Such long physical half-lives allow time for the antibodies to accumulate in the tumor while simultaneously preventing the premature decay of their radioactive payload. However, this combination of long biologic and physical half-lives gives rise to a critical limiting complication: high activity concentrations in and radiation doses to non-target organs.

To circumvent this problem, considerable attention has been dedicated to the development of targeting methodologies that combine the advantages of antibodies with the pharmacokinetics of smaller molecules. One particularly appealing method of achieving this balance while still using intact antibodies is termed pretargeting [3-5]. Generally, pre-targeted methodologies involve 4 steps: the injection into the bloodstream of a bivalent antibody with the ability to bind both an antigen and a radioligand; the slow accumulation of the antibody in the tumor and concomitant clearance of the antibody from the blood; the injection into the bloodstream of the small-molecule radioligand; and the binding of the radioligand to the antibody, followed by the rapid clearance of excess radioactivity. The pharmacokinetics of the small-molecule ligands not only reduces background radiation dose to non-target organs but also facilitates the use of radioisotopes with short half-lives that would normally be incompatible with antibody-based imaging.

Two strategies for pre-targeted systems predominate in the literature. In the first, bispecific antibodies capable of binding both an antigen and a radiolabeled hapten—such as a diethylenetriaminepentaacetic acid-chelated radiometal or a chelator-modified biotin—are used [6,7]. In the second strategy, antibodies covalently modified with oligomeric nucleic acids are used as the targeting vectors while radiolabeled, complementary oligonucleotide sequences are used as the radioligands [8]. Both systems are creative solutions and have proven successful in vivo [9, 10]. Each, however, possesses significant limitations that threaten their clinical applicability. For the former, these include the complexity and expense associated with the generation of bispecific antibodies and the immunogenicity of streptavidin-based systems; for the latter, both the in vivo stability of the olignucleotides and the expense of using non-natural nucleic acid analogs are concerns.

In recent years, a chemical methodology has been developed that has the potential to aid in the creation of novel, antibody-based pre-targeting systems without these limitations: bioorthogonal click chemistry [11]. Although the [3+2] Huisgen cycloaddition between an azide and alkyne remains the preeminent form of click chemistry [12], another promising click ligation variant has recently garnered considerable interest: the rapid, selective, and bioorthogonal inverse electron-demand [4+2] Diels-Alder cycloaddition between a tetrazine and a strained alkene dienophile (FIG. 12) [13,14]. To date, the ligation has been used in a variety of settings, including fluorescence imaging with antibodies and nanoparticles [13] and PET imaging with both ¹⁸F- and radiometal-based probes [15-17]. Recently, and most applicable here, 2 reports have been made of pre-targeting systems based on the tetrazine-dienophile ligation: one using a small molecule tetrazine moiety equipped with a SPECT isotope and the second using a larger, tetrazine-modified, ¹⁸F-labeled dextran-based radioligand [18,19].

Herein, we report the development of a pre-targeted PET imaging methodology based on the [4+2] cycloaddition reaction between tetrazine and transcyclooctene (FIG. 13). For proof of concept, we have used a colorectal cancer cell line, the A33 antibody, and the positron-emitting radiometal ⁶⁴Cu as a model system. Ultimately, we have found that the in vivo click methodology is highly effective at delineating tumor from normal tissue, producing PET images with contrast comparable to that obtained with directly radiolabeled antibodies.

Ultimately, an in vivo comparison with the preassembled and purified ⁶⁴Cu-NOTA-A33 construct is essential for the evaluation of the efficacy of this pre-targeted system. To this end, the ⁶⁴Cu-labeled antibody was synthesized as described above and purified to more than 99% radiochemical purity, with a specific activity of 125.8±11.1 MBq/mg and an immunoreactivity of more than 95%. In the biodistribution experiment, nude mice bearing SW1222 xenografts in the right shoulder were injected with ⁶⁴Cu-NOTA-A33 (0.55-0.75 MBq, 4-6 mg) and were euthanized at 4, 12, 24, and 48 h, followed by the collection and weighing of tissues and assay of ⁶⁴Cu activity in each tissue.

High specific uptake of the radiotracer was observed in the SW1222 xenografts, with the % ID/g increasing from 18.2±3.0 at 4 h to 35.0±3.8 at 48 h. As is typical of antibody-based imaging, a concomitant decrease in the % ID/g in the blood also occurred over the course of the experiment. The organs with the highest background uptake were the lungs, liver, and spleen, though the uptake in these organs was at its highest point at 4 h; by 48 h, the tumor-to-organ ratios for each of these organs were 20.8±13.1, 10.0±4.5, and 19.8±5.4, respectively.

These biodistribution data were corroborated by small-animal PET imaging. In these experiments, tumor-bearing mice were injected with 64Cu-NOTA-A33, and PET images were acquired at various time points between 4 and 48 h. The results clearly indicate that the ⁶⁴Cu-NOTA-A33 constructs are taken up in the antigen-expressing SW1222 tumor (FIG. 18A). High blood-pool activity and background uptake are evident at early time points, but over the course of the experiment, the tumor signal increases significantly to a point at which it is by far the most prominent feature in the image.

As expected for an intact antibody, the ⁶⁴Cu-NOTA-A33 displays higher absolute uptake in the tumor than the pre-targeted system: 33.1±7.0% ID/g at 24 h, compared with 4.0±0.9% ID/g for the click chemistry approach at the same time point. However, for imaging purposes, the far more important question is target-to-background ratio, and indeed, when speaking in terms of relative uptake, the methodologies become far more similar. At 24 h after injection, the pre-targeting methodology provides a tumor-to-muscle activity ratio that is statistically identical to that of ⁶⁴Cu-NOTA-A33: 27.0±7.4 versus 33.8±6.7, respectively. In addition, at all time points assayed, the uptake in non-target tissues is significantly higher for the intact antibody than for the pre-targeting system in all organs save the large intestine.

Not surprisingly given the pharmacokinetics of intact antibodies, at later time points, the tumor-to-muscle activity ratio for the intact ⁶⁴Cu-NOTA-A33 surpasses the maximum values measured for the pre-targeting system, reaching 52.2±14.7 at 48 h. Likewise, whereas the tumor-to-blood ratios of 64Cu-NOTA-A33 and the pre-targeting system are comparable at 24 h (2.9±0.4 and 1.9±0.6, respectively), at 48 h, the tumor-to-blood ratio of ⁶⁴Cu-NOTA-A33 (24.5±11.6) exceeds the maximum ratio observed with the pre-targeting system. Yet despite the high absolute and relative uptake of ⁶⁴Cu-NOTA-A33 at these later time points, the data clearly illustrate that the pre-targeted system represents a qualitatively and quantitatively comparable alternative to ⁶⁴Cu-based antibody imaging.

The preceding comparison to ⁶⁴Cu-NOTA-A33 undoubtedly provides the most direct measure of the comparative efficacy of the pretargeting system. However, the recent and rapid advent of clinical ⁸⁹Zr-immunoPET likely means that many of the antibody-based imaging agents in the future will be labeled with ⁸⁹Zr. Thus, we believe that a comparison to directly labeled ⁸⁹Zr-DFO-A33 could also be valuable by providing insight into the merits of ⁶⁴Cu-based pre-targeting as an alternative to antibodies labeled with ⁸⁹Zr. To this end, ⁸⁹Zr-DFO-A33 was synthesized by modifying A33 with the ⁸⁹Zr chelator desferrioxamine via isothiocyanate coupling (3.5±1.1 DFO/A33) and radiolabeling this construct with ⁸⁹Zr using standard procedures [2]. The purified radiopharmaceutical exhibited more than 99% radiochemical purity, an immunoreactivity of 92%±5%, more than 95% stability over 7 d at 37° C., and a specific activity of 159.1±22.2 MBq/mg.

For both PET imaging and biodistribution experiments, SW1222 tumor-bearing animals were injected with ⁸⁹Zr-DFO-A33 (10.2-12.0 MBq for PET, 0.55-0.75 MBq for biodistribution) and subsequently imaged or euthanized for biodistribution at time points between 4 and 120 h after injection. Both the PET and the biodistribution experiments display all the hallmarks of ⁸⁹Zr-based antibody imaging (FIG. 18B). High early levels of activity in the blood (58.4±8.5% ID/g at 4 h) decrease over the first 48 h as the activity levels in the tumor increase dramatically to a maximum of 44.7±10.5% ID/g at 48 h. All other tissues exhibit much lower activities after 48 h with the exception of bone, which displays uptake values of approximately 10% ID/g, a consequence of the mineralization of liberated ⁸⁹Zr4 in bone.

In comparative terms, the directly labeled ⁸⁹Zr-DFO-A33, like ⁶⁴Cu-NOTA-A33, is characterized by higher absolute tumor uptake, with 43.3±9.0% ID/g in the xenograft at 24 h compared with 4.0±0.9% ID/g for the pre-targeting system. Again, however, the pre-targeting methodology excels with regard to the more important metric: tumor-to-background ratio. Here, the pre-targeting approach produces a higher tumor-to-muscle activity ratio than ⁸⁹Zr-DFO-A33 at 24 h after injection (27.0±7.4 vs. 12.7±3.3, respectively) as well as a comparable tumor-to-blood activity ratio at the same time point (1.9±0.6 vs. 2.4±0.7, respectively). Further, the background associated with the intact antibody is significantly higher than in the pre-targeted system.

Not surprisingly, though, both the absolute uptake and the tumor-to-background ratios for ⁸⁹Zr-DFO-A33 increase further at later time points. For example, despite the higher tumor-to-muscle ratio produced by the pre-targeting approach at 24 h, the tumor-to-muscle activity ratios for ⁸⁹Zr-DFO-A33 surpass this value at later time points, ultimately reaching a maximum of 57.7±7.0 at 72 h.

Critically, for both ⁶⁴Cu-NOTA-A33 and ⁸⁹Zr-DFO-A33, the high absolute uptake values and tumor-to-background ratios observed at later time points do not come without cost. In both cases—⁸⁹Zr, in particular—the pharmacokinetics of the antibodies mean that high levels of radioactivity persist in the patient for extended periods of time; although these levels of activity are often highest in the tumor, the levels in background organs are relatively high as well. It is in this regard that the pre-targeted strategy offers an exciting and innovative alternative, for the data clearly indicate that the pre-targeted approach produces images comparable to those created with directly radiolabeled antibodies but with both accelerated pharmacokinetics and dramatically lower background uptake in non-target tissues.

This discussion leads us to another potential advantage to consider for the pre-targeting system: reduced radiation dose to non-target tissues. Using biodistribution data, we performed dosimetry calculations for the pre-targeting system, ⁶⁴Cu-NOTA-A33, and ⁸⁹Zr-DFO-A33 using the OLINDA computer program to determine mean organ absorbed doses and effective dose in mGy/MBq and mSv/MBq respectively (Table 2) [24]. The data indicate that the pre-targeting system provides a significant dosimetric advantage, compared with the ⁸⁹Zr-labeled antibody: the effective dose with ⁶⁴Cu pre-targeting is 0.0124 mSv/MBq whereas that due to ⁸⁹Zr-DFO-A33 is 0.416 mSv/MBq. A more pronounced disparity between the two exists when considering the mean absorbed dose to the bone, specifically red marrow and osteogenic cells; with ⁸⁹Zr-DFO-A33, the dose to these tissues is 0.843 and 1.646 mGy/MBq, respectively, compared with 0.0413 and 0.0230 with 64Cu pre-targeting. Not surprisingly given the shorter half-life of ⁶⁴Cu, these advantages are not as pronounced when comparing the pre-targeting system to ⁶⁴Cu-NOTA-A33. The effective dose of ⁶⁴Cu-NOTA-A33 is 0.0359 mSv/MBq, approximately 3 times larger than the pre-targeting value of 0.0124 mSv/MBq. Thus, whereas the dosimetric differences are somewhat attenuated when making comparisons to the ⁶⁴Cu-labeled antibody, it becomes clear that the pre-targeting system provides qualitatively and quantitatively comparable imaging results at only a fraction of the background radiation dose of the directly labeled antibodies, particularly the ⁸⁹Zr-labeled construct.

Herein, we have reported the development of a modular pre-targeting system for PET imaging based on the inverse electron-demand Diels-Alder cycloaddition between tetrazine and transcyclooctene. In a model system, we have found that this in vivo click methodology produces high-quality images with tumor-to-background contrast comparable to that achieved with directly radiolabeled antibodies. Further, the system facilitates the production of these images at a dramatically reduced absorbed dose to non-target organs compared with imaging with a ⁸⁹Zr-labeled antibody.

Despite these successes, the system is not without slight limitations. First, the system requires that the antibody either be non-internalizing or have a slow rate of internalization; although this applies to both A33 and several other clinically interesting antibodies (e.g., CC49), there are certainly other clinically relevant antibodies that are known to internalize upon antigen binding and thus would be much more challenging to use in a pre-targeted strategy. And second, the relatively slow fecal excretion of the excess radioligand limits both the ability to image abdominal tumors rapidly after the administration of the radioligand and the clinical use of radionuclides with short half-lives, such as ¹⁸F or ⁶⁸Ga. Currently, experiments are under way to develop novel tetrazine constructs that have altered pharmacokinetics and pharmacodynamics and thus should enable more rapid imaging. However, these changes may not be strictly necessary in the clinic. If other methods for clearing radioactive feces from the patient are adopted, images can be recorded at earlier time points, and radionuclides with short half-lives such as ¹⁸F or ⁶⁸Ga may be used, further augmenting the dosimetric benefits of the approach. Ultimately, we believe that this pre-targeted PET imaging methodology could be an extremely valuable and versatile clinical tool. Specifically, the success of the A33-based proof-of-concept system described here—both in terms of tumor delineation and in terms of dosimetry—has identified the system as a prime candidate for translation to the clinic for the imaging of colorectal cancer. Translation of this approach is an enticing prospect for two principal reasons. First, from a dosimetry perspective, the 64Cu/A33 pre-targeting system would almost certainly prove a safer alternative for patients than the ²⁴I-A33 PET radiotracer currently used, without undue sacrifice in image quality. Second, the targeting of A33 to normal bowel has proved a major concern in clinical trials using A33 [25]. Kinetic models, however, have suggested that the half-life of the antibody in normal colon is far less than that of the antibody in the tumor. A pre-targeting strategy would be a near ideal way to circumvent this complication, because the pre-targeted radioligand could be injected after the shedding of the antibody from the normal colon but before the departure of the antibody from the cancerous tissue.

Equally important, this methodology could also have a significant impact beyond the system described here. Along these lines, the modularity of the system is an incredibly important asset: the transcyclooctene moiety can be appended to any antibody of interest, and the simplicity of the tetrazine-based radioligand facilitates the creation of a library of constructs bearing various radioisotopes. Indeed, preliminary experiments are under way in the creation of pre-targeting systems for both ⁸⁹Zr-based PET imaging and ¹⁷⁷Lu- and ⁹⁰Y-based radiotherapy. However, the paramount value of the system plainly lies in its ability to harness the advantages of antibody-based radiopharmaceuticals while skirting their pharmacokinetic limitations. The methodology produces high tumor-to-background contrast at relatively early time points while limiting uptake in, and thus dose to, normal organs, especially compared with antibodies bearing radionuclides with long physical half-lives. Consequently, this approach may ultimately offer an effective and safer alternative to both immunoPET and immunoSPECT imaging with long-lived isotopes such as ⁸⁹Zr, ¹¹¹In, or ¹²⁴I and radioimmunotherapy with isotopes such as ¹⁷⁷Lu, ⁹⁰Y, or ¹³¹I.

### REFERENCES

1. Wuts, P. G. M. and Greene, T. W. (2006) Greene's Protective Groups in Organic Synthesis, 4th ed., John Wiley & Sons, Inc., Hoboken, N.J.
2. Fleisher, D., Bong, R., and Stewart, B. H. (1996) Improved oral drug delivery: solubility limitations overcome by the use of prodrugs, Adv. Drug Deliv. Rev. 19(2), 115-130.
3. Li, Z. et al. (2010) Tetrazine-trans-cyclooctene ligation for the rapid construction of 18F labeled probes, Chemical Communications 46(42), 8043-8045.
4. Keliher, E. J. et al. (2011) High-Yielding, Two-Step 18F Labeling Strategy for 18F-PARP1 Inhibitors, ChemMedChem 6(3), 424-427.
5. Zeglis, B. M. et al. (2011) Modular Strategy for the Construction of Radiometalated Antibodies for Positron Emission Tomography Based on Inverse Electron Demand Diels-Alder Click Chemistry, Bioconjug. Chem. 22(10), 2048-2059.
6. Collins, J. M., Lambert, J. J., and Collins, M. J. “Microwave-assisted peptide synthesis,” U.S. Pat. No. 7,393,920 application Ser. No. 10/604,022, filed Jun. 23, 2003. (Published Jul. 1, 2008).
7. Collins, J. M. “Microwave enhanced N-fmoc deprotection in peptide synthesis,” U.S. Pat. No. 8,314,208 application Ser. No. 11/351,796, filed Feb. 10, 2006. (Published Nov. 20, 2012).
8. Boerman, O. and Gotthardt, M. (2012) 18F-Labelled exendin to image GLP-1 receptor-expressing tissues: from niche to blockbuster?, Eur. J. Nucl. Med. Mol. Imaging 39(3), 461-462.
9. Alauddin, M. M. (2012) Positron emission tomography (PET) imaging with ¹⁸F-based radiotracers, American Journal of Nuclear Medicine and Molecular Imaging 2(1), 55-76.
10. Guhlke, S. et al. (1994) (2-[18F]fluoropropionyl-(d)phe1)-octreotide, a potential radiopharmaceutical for quantitative somatostatin receptor imaging with PET: Synthesis, radiolabeling, in vitro validation and biodistribution in mice, Nucl. Med. Biol. 21(6), 819-825.
11. Fani, M. et al. (2011) PET of Somatostatin Receptor-Positive Tumors Using 64Cu- and 68Ga-Somatostatin Antagonists: The Chelate Makes the Difference, J. Nucl. Med. 52(7), 1110-1118.
12. Zhang, X. et al. (2006) 18F-Labeled Bombesin Analogs for Targeting GRP Receptor-Expressing Prostate Cancer, J. Nucl. Med. 47(3), 492-501.
13. Honer, M. et al. (2011) 18F-Labeled Bombesin Analog for Specific and Effective Targeting of Prostate Tumors Expressing Gastrin-Releasing Peptide Receptors, J. Nucl. Med. 52(2), 270-278.
14. Yang, M. et al. (2011) ¹⁸F-Labeled GRPR Agonists and Antagonists: A Comparative Study in Prostate Cancer Imaging, Theranostics 1, 220-229.
15. Lears, K. A. et al. (2011) In Vitro and In Vivo Evaluation of ⁶⁴Cu-Labeled SarAr-Bombesin Analogs in Gastrin-Releasing Peptide Receptor-Expressing Prostate Cancer, J. Nucl. Med. 52(3), 470-477.
16. Kiesewetter, D. et al. (2012)¹⁸F-radiolabeled analogs of exendin-4 for PET imaging of GLP-1 in insulinoma, Eur. J. Nucl. Med. Mol. Imaging 39(3), 463-473.
17. Wang, Y. et al. (2012) Synthesis and evaluation of [¹⁸F]exendin (9-39) as a potential biomarker to measure pancreatic β-cell mass, Nucl. Med. Biol. 39(2), 167-176.
18. Wu, Z. et al. (2011) In Vivo Imaging of Transplanted Islets with ⁶⁴Cu-DO3A-VS-Cys40-Exendin-4 by Targeting GLP-1 Receptor, Bioconjug. Chem. 22(8), 1587-1594.
19. Hennig, I. M. et al. (1995) Substance-P receptors in human primary neoplasms: tumoral and vascular localization, Int. J. Cancer 61(6), 786-792.
20. Reubi, J. C. (2003) Peptide Receptors as Molecular Targets for Cancer Diagnosis and Therapy, Endocr. Rev. 24(4), 389-427.
21. Devaraj, N. K. et al. (2010) Bioorthogonal Turn-On Probes for Imaging Small Molecules inside Living Cells, “Angew. Chem., Int. Ed.” 49(16), 2869-2872.
22. Reiner, T. et al. (2010) Bioorthogonal Small-Molecule Ligands for PARP1 Imaging in Living Cells, ChemBioChem 11(17), 2374-2377.
23. Chung, H. J. et al. (2011) Ubiquitous Detection of Gram-Positive Bacteria with Bioorthogonal Magnetofluorescent Nanoparticles, ACS Nano 5(11), 8834-8841.
24. Ullal, A. V. et al. (2011) Nanoparticle-Mediated Measurement of Target-Drug Binding in Cancer Cells, ACS Nano 5(11), 9216-9224.
25. Haun, J. B. et al. (2011) Micro-NMR for Rapid Molecular Analysis of Human Tumor Samples, Science Translational Medicine 3(71), 71ra16.
26. Reiner, T. et al. (2011) Synthesis and In Vivo Imaging of a ¹⁸F-Labeled PARP1 Inhibitor Using a Chemically Orthogonal Scavenger-Assisted High-Performance Method, “Angew. Chem., Int. Ed.” 50(8), 1922-1925.
27. Devaraj, N. K. and Weissleder, R. (2011) Biomedical Applications of Tetrazine Cycloadditions, Acc. Chem. Res. 44(9), 816-827.
28. Reiner, T. et al. (2012) Imaging Therapeutic PARP Inhibition In Vivo through Bioorthogonally Developed Companion Imaging Agents, Neoplasia 14(3), 169-177.
29. Grossman, S. A. and Batara, J. F. (2004) Current management of glioblastoma multiforme, Semin. Oncol. 31(5), 635-644.
30. Staquicini, F. I. et al. (2011) Systemic combinatorial peptide selection yields a non-canonical iron-mimicry mechanism for targeting tumors in a mouse model of human glioblastoma, J. Clin. Invest. 121(1), 161-173.
31. Keliher, E. J. et al. (2012) Efficient 18F-Labeling of Synthetic Exendin-4 Analogues for Imaging Beta Cells, ChemistryOpen 1(4), 177-183.
32. Selvaraj, R. et al. (2011) Tetrazine-trans-cyclooctene ligation for the rapid construction of integrin αvβ3 targeted PET tracer based on a cyclic RGD peptide, Bioorg. Med. Chem. Lett. 21(17), 5011-5014.
33. Reiner, T. et al. (2011) Accurate measurement of pancreatic islet β-cell mass using a second-generation fluorescent exendin-4 analog, Proc. Natl. Acad. Sci. U.S.A 108(31), 12815-12820.
34. Reiner, T. et al. (2010) Near-Infrared Fluorescent Probe for Imaging of Pancreatic β Cells, Bioconjug. Chem. 21(7), 1362-1368.
35. Sletten, E. M. and Bertozzi, C. R. (2009) Bioorthogonal Chemistry: Fishing for Selectivity in a Sea of Functionality, “Angew. Chem., Int. Ed.” 48(38), 6974-6998.
36. Valkó, K., Bevan, C., and Reynolds, D. (1997) Chromatographic Hydrophobicity Index by Fast-Gradient RP-HPLC: A High-Throughput Alternative to log P/log D, Anal. Chem. 69(11), 2022-2029.
37. Leyton, J. et al. (2011) Targeting Somatostatin Receptors: Preclinical Evaluation of Novel 18F-Fluoroethyltriazole-Tyr3-Octreotate Analogs for PET, J. Nucl. Med. 52(9), 1441-1448.
38. Lal, S. et al. (2000) An implantable guide-screw system for brain tumor studies in small animals, J. Neurosurg. 92(2), 326-333.
39. Taylor, J. E. et al. (1994) Detection of somatostatin receptor subtype 2 (SSTR2) in established tumors and tumor cell lines: Evidence for SSTR2 heterogeneity, Peptides 15(7), 1229-1236.
40. Yang, J. et al. (2012) Metal-Catalyzed One-Pot Synthesis of Tetrazines Directly from Aliphatic Nitriles and Hydrazine, “Angew. Chem., Int. Ed.” 51(21), 5222-5225.
41. Devaraj, N. K. et al. (2009) Fast and Sensitive Pretargeted Labeling of Cancer Cells through a Tetrazine/trans-Cyclooctene Cycloaddition, “Angew. Chem., Int. Ed.” 48(38), 7013-7016.

### ADDITIONAL REFERENCES

1. Wu, A. M., Journal of Nuclear Medicine 2009, 50 (1), 2-5.
2. Zeglis, B.; Lewis, J. S., Dalton Transactions 2011.
3. Sharkey, R. M.; Chang, C. H.; Rossi, E. A.; McBride, W. J.; Goldenberg, D. M., Tumor Biology 2012, 33, 591-600.
4. Hollander, N., Immunotherapy 2009, 1 (2), 211-222.
5. He, J.; Liu, G; Gupta, S.; Zhang, H.; Rusckowski, M.; Hnatowich, D., Journal of Nuclear Medicine 2004, 45, 1087-1095.
6. Goldenberg, D. M.; Sharkey, R. M.; Paganelli, G; Barbet, J.; Chatal, J. F., J. Clin. Oncol. 2006, 24 (5), 823-834.
7. Boerman, 0. C.; van Schaijk, F. G; Oyen, W. J. G; Corstens, F. H. M., Journal of Nuclear Medicine 2003, 44 (3), 400-411.
8. Sharkey, R. M.; Karacay, H.; Cardllo, T. M.; Chang, C. H.; McBride, W. J.; Rossi, E. A.; Horak, I. D.; Goldenberg, D. M., Clin. Cancer Res. 2005, 11 (19), 7109s-7121s.
9. Sharkey, R. M.; Goldenberg, D. M., Cancer Investigation 2006, 24 (1), 82-97.
10. Walter, R. B.; Press, 0. W.; Pagel, J. M., Cancer Biother Radiopharm. 2010, 25 (2), 125-142.
11. Blackman, M. L.; Royzen, M.; Fox, J. M., Journal of the American Chemical Society 2008, 130, 13518-13519.
12. Karver, M. R.; Weissleder, R.; Hilderbrand, S. A., Bioconjugate Chemistry 2011, 22 (11), 2263-2270.
13. Boger, D. L., Chemical Reviews 1986, 86, 781-793.
14. Liu, D. S.; Tangpeerachaikul, A.; Selvaraj, R.; Taylor, M. T.; Fox, J. M.; Ting, A. Y., Journal of the American Chemical Society 2012, 134, 792-795.
15. Selvaraj, R.; Liu, S. L.; Hassink, M.; Huang, C. W.; Yap, L. P.; Park, R.; Fox, J. M.; Li, Z. B.;
Conti, P. S., Bioorg. Med. Chem. Lett. 2011, 21 (17), 5011-5014.
16. Reiner, T.; Keliher, E. J.; Earley, S.; Marinelli, B.; Weissleder, R., Angewandte Chemie International Edition 2011, 50, 1922-1925.
17. Zeglis, B. M.; Mohindra, P.; Weissmann, G. I.; Divilov, V.; Hilderbrand, S. A.; Weissleder, R.; Lewis, J. S., Bioconjugate Chemistry 2011, 22 (10), 2048-2059.
18. Devaraj, N. K.; Thurber, G. M.; Keliher, E. J.; Marinelli, B.; Weissleder, R., Proceedings of the National Academy of Sciences of the U.S.A. 2012, 109 (13), 4762-4767.
19. Li, Z.; Cai, H.; Hassink, M.; Blackman, M. L.; Brown, R. C. D.; Conti, P. S.; Fox, J. M., Chemical Communications 2010, 46 (42), 8043-8045.
20. Rossin, R.; Verkerk, P. R.; van den Bosch, S. M.; Vulders, R. C. M.; Verel, I.; Lub, J.; Robillard, M. S., Angewandte Chemie International Edition 2010, 49, 3375-3378.
21. Zeglis, B. M.; Sevak, K. K.; Reiner, T.; Mohindra, P.; Carlin, S.; Zanzonico, P.; Weissleder, R.; Lewis, J. S., Journal of Nuclear Medicine 2013, 54, 1389-1396.
22. Zeng, D.; Zeglis, B. M.; Lewis, J. S.; Anderson, C. J., Journal of Nuclear Medicine 2013, Ahead of Print.
23. Sakamoto, J.; Kojima, H.; Kato, J.; Hamashima, H.; Suzuki, H., Cancer Chemotherapy and Pharmacology 2000, 46, S27-S32.
24. Scott, A. M.; Lee, F. T.; Jones, R.; Hopkins, W.; MacGregor, D.; Cebon, J. S.; Hannah, A.; Chong, G; U, P.; Papenfuss, A.; Rigopoulos, A.; Sturrock, S.; Murphy, R.; Wirth, V.; Murone, C.; Smyth, F. E.; Knight, S.; Welt, S.; Ritter, G; Richards, E.; Nice, E. C.; Burgess, A. W.; Old, L. J., Clin. Cancer Res. 2005, 11 (13), 4810-4817.
25. Ackerman, M. E.; Chalouni, C.; Schmidt, M. M.; Raman, V. V.; Ritter, G; Old, L. J.; Mellman, I.; Wittrup, K. D., Cancer Immunology and Immunotherapy 2008, 57, 1017-1027.
26. Carrasquillo, J. A.; Pandi-Taskar, N.; O'Donoghue, J. A.; Humm, J. L.; Zanzonico, P.; Smith-Jones, P. M.; Divgi, C. R.; Pryma, D. A.; Ruan, S.; Kemeny, N. E.; Fong, Y; Wong, D.; Jaggi, J. S.; Scheinberg, D. A.; Gonen, M.; Panageas, K. S.; Ritter, G; Jungbluth, A. A.; Old, L. J.; Larson, S. M., Journal of Nuclear Medicine 2011, 52, 1173-1180.
27. Welt, S.; Scott, A. M.; Divgi, C. R.; Kemeny, N. E.; Finn, R. D.; Daghighian, F.; StGermain, J.; Richards, E. C.; Larson, S. M.; Old, L. J., J. Clin. Oncol. 1996, 14 (6), 1787-1797.

### ADDITIONAL REFERENCES

1. Wu A M. Antibodies and antimatter: the resurgence of immuno-PET. J Nucl Med. 2009; 50:2-5.
2. Zeglis B, Lewis J S. A practical guide to the construction of radiometallation bioconjugates for positron emission tomography. Dalton Trans. 2011; 40: 6168-6195.
3. Hollander N. Bispecific antibodies for cancer therapy. Immunotherapy. 2009; 1: 211-222.
4. Sharkey R M, Goldenberg D M. Advances in radioimmunotherapy in the age of molecular engineering and pretargeting. Cancer Invest. 2006; 24:82-97.
5. Boerman O C, van Schaijk F G, Oyen W J G, Corstens F H M. Pretargeted radio [symbol] immunotherapy of cancer: progress step by step. J Nucl Med. 2003; 44:400-411.
6. Press O W, Corcoran M, Subbiah K, et al. A comparative evaluation of conventional and pretargeted radioimmunotherapy of CD20-expressing lymphoma xenografts. Blood. 2001; 98:2535-2543.
7. Goldenberg D M, Chang C H, Rossi E A, McBride W J, Sharkey R M. Pretargeted molecular imaging and radioimmunotherapy. Theranostics. 2012; 2:523-540.
8. Liu G, Dou S, Liu Y, Wang Y, Rusckowski M, Hnatowich D J. 90Y-labeled phosphorodiamidate morpholino oligomer for pretargeting radiotherapy. Biocon [symbol] jug Chem. 2011; 22:2539-2545.
9. Lewis M R, Wang M, Axworthy D B, et al. In vivo evaluation of pretargeted 64Cu for tumor imaging and therapy. J Nucl Med. 2003; 44:1284-1292.
10. Mohsin H, Jia F, Bryan J N, et al. Comparison of pretargeted and conventional CC49 radioimmunotherapy using 149Pm, 166Ho, and 177Lu. Bioconjug Chem. 2011; 22:2444-2452.
11. Kolb H C, Finn M G, Sharpless K B. Click chemistry: diverse chemical function from a few good reactions. Angew Chem Int Ed Engl. 2001; 40:2004-2021.
12. Sletten E M, Bertozzi C R. Bioorthogonal chemistry: fishing for selectivity in a sea of functionality. Angew Chem Int Ed Engl. 2009; 48:6973-6998.
13. Devaraj N K, Upadhyay R, Hatin J B, Hilderbrand S A, Weissleder R. Fast and sensitive pretargeted labeling of cancer cells through a tetrazine/trans-cyclooctene cycloaddition. Angew Chem Int Ed Engl. 2009; 48:7013-7016.
14. Blackman M L, Royzen M, Fox J M. Tetrazine ligation: fast bioconjugation based on inverse electron demand Diels-Alder reactivity. J Am Chem Soc. 2008; 130: 13518-13519.
15. Zeglis B M, Mohindra P, Weissmann G I, et al. Modular strategy for the construc [symbol] tion of radiometalated antibodies for positron emission tomography based on inverse electron demand Diels-Alder click chemistry. Bioconjug Chem. 2011; 22: 2048-2059.
16. Li Z, Cai H, Hassink M, et al. Tetrazine-trans-cyclooctene ligation for the rapid construction of 18-F labeled probes. Chem Commun (Camb). 2010; 46:8043-8045.
17. Reiner T, Keliher E J, Earley S, Marinelli B, Weissleder R. Synthesis and in vivo imaging of a 18F-labeled PARP1 inhibitor using a chemically orthogonal scavenger-assisted high-performance method. Angew Chem Int Ed Engl. 2011; 50:1922-1925.
18. Rossin R, Verkerk P R, van den Bosch S M, et al. In vivo chemistry for pretargeted tumor imaging in live mice. Angew Chem Int Ed Engl. 2010; 49:3375-3378.
19. Devaraj N K, Thurber G M, Keliher E J, Marinelli B, Weissleder R. Reactive polymer enables efficient in vivo bioorthogonal chemistry. Proc Natl Acad Sci USA. 2012; 109:4762-4767.
20. Carrasquillo J A, Pandi-Taskar N, O'Donoghue J A, et al. 124I-huA33 antibody PET of colorectal cancer. J Nucl Med. 2011; 52:1173-1180.
21. Ackerman M E, Chalouni C, Schmidt M M, et al. A33 antigen displays persistent surface expression. Cancer Immunol Immunother. 2008; 57:1017-1027.
22. Wadas T J, Wong E H, Weisman G R, Anderson C J. Coordinating radiometals of copper, gallium, indium, yttrium, and zirconium for PET and SPECT imaging of disease. Chem Rev. 2010; 110:2858-2902.
23. Heneweer C, Holland J P, Divilov V, Carlin S, Lewis J S. Magnitude of enhanced permeability and retention effect in tumors with different phenotypes: Zr-89-albumin as a model system. J Nucl Med. 2011; 52:625-633.
24. Stabin M G, Sarks R B, Crowe E. OLINDA/EXM: the second-generation personal computer software for internal dose assessment in nuclear medicine. J Nucl Med. 2005; 46:1023-1027.
25. Welt S, Scott A M, Divgi C R, et al. Phase I/II study of iodine 125-labeled monoclonal antibody A33 in patients with advanced colon cancer. J Clin Oncol. 1996; 14:1787-1797.

TABLE 1
Biodistribution Data for In Vivo ⁶⁴Cu Pretargeting Experiment

| Organ | 1 h | 4 h | 12 h | 24 h |
|---|---|---|---|---|
| Blood | 3.5 ± 0.6 | 2.6 ± 0.8 | 2.3 ± 0.4 | 2.1 ± 0.5 |
| Tumor | 4.1 ± 0.3 | 4.1 ± 0.6 | 4.2 ± 0.8 | 4.0 ± 0.9 |
| Heart | 1.1 ± 0.2 | 0.9 ± 0.3 | 0.9 ± 0.1 | 0.8 ± 0.2 |
| Lung | 1.6 ± 0.5 | 1.6 ± 0.4 | 1.1 ± 0.4 | 1.0 ± 0.3 |
| Liver | 2.2 ± 0.3 | 1.3 ± 0.3 | 0.9 ± 0.2 | 1.1 ± 0.2 |
| Spleen | 0.6 ± 0.1 | 0.5 ± 0.2 | 0.6 ± 0.3 | 0.4 ± 0.1 |
| Stomach | 0.5 ± 0.1 | 0.3 ± 0.1 | 0.5 ± 0.6 | 0.2 ± 0.0 |
| Large intestine | 0.5 ± 0.0 | 3.1 ± 1.1 | 2.3 ± 1.0 | 1.1 ± 0.4 |
| Feces | 11.9 ± 4.4 | 8.8 ± 3.4 | 2.5 ± 0.1 | 1.4 ± 0.5 |
| Small intestine | 0.0 ± 0.0 | 0.4 ± 0.1 | 0.8 ± 0.5 | 0.3 ± 0.0 |
| Kidney | 1.3 ± 0.1 | 0.9 ± 0.3 | 0.9 ± 0.3 | 0.7 ± 0.2 |
| Muscle | 0.2 ± 0.0 | 0.1 ± 0.0 | 0.2 ± 0.0 | 0.1 ± 0.0 |
| Bone | 0.3 ± 0.2 | 0.3 ± 0.2 | 0.4 ± 0.1 | 0.3 ± 0.1 |
| Skin | 0.3 ± 0.1 | 0.3 ± 0.1 | 0.4 ± 0.1 | 0.5 ± 0.2 |

Values are % ID/g ± SD. Mice (n = 4) bearing subcutaneous SW1222 xenografts were administered A33-TCO (100 μg) via tail vein injection. After 24 h, the same mice were administered ⁶⁴Cu-Tz-Bn-NOTA (0.55-0.75 MBq [15-20 μCi]) via tail vein injection (t = 0).

TABLE 2
Dosimetry Calculations for Imaging Constructs Studied

| | ⁶⁴Cu | ⁶⁴Cu- | ⁸⁹Zr- |
|---|---|---|---|
| Target organ* | pretargeting^{†} | NOTA-A33^{‡} | DFO-A33^{§} |
| Adrenals | 0.0068 | 0.0196 | 0.4432 |
| Brain | 0.0064 | 0.0150 | 0.2065 |
| Breasts | 0.0056 | 0.0138 | 0.1678 |
| Gallbladder wall | 0.0074 | 0.0200 | 0.3892 |
| Lower large intestine Wall | 0.0449 | 0.0522 | 0.3622 |
| Small intestine | 0.0089 | 0.0225 | 0.3000 |
| Stomach wall | 0.0072 | 0.0239 | 0.2565 |
| Upper large intestine wall | 0.0308 | 0.0392 | 0.3243 |
| Heart wall | 0.0079 | 0.0292 | 0.4189 |
| Kidneys | 0.0085 | 0.0503 | 0.6838 |
| Liver | 0.0084 | 0.0524 | 0.7676 |
| Lungs | 0.0078 | 0.0484 | 0.6108 |
| Muscle | 0.0037 | 0.0148 | 0.3432 |
| Ovaries | 0.0081 | 0.0184 | 0.2946 |
| Pancreas | 0.0070 | 0.0191 | 0.3703 |
| Red marrow | 0.0143 | 0.0832 | 0.8432 |
| Osteogenic cells | 0.0230 | 0.1186 | 1.6459 |
| Skin | 0.0052 | 0.0125 | 0.1830 |
| Spleen | 0.0049 | 0.0324 | 0.6811 |
| Testes | 0.0061 | 0.0141 | 0.1846 |
| Thymus | 0.0061 | 0.0158 | 0.2670 |
| Thyroid | 0.0062 | 0.0152 | 0.2559 |
| Urinary bladder wall | 0.0071 | 0.0165 | 0.2232 |
| Uterus | 0.0075 | 0.0176 | 0.2543 |
| Total body | 0.0074 | 0.0231 | 0.3757 |
| Effective dose | 0.0124 | 0.0359 | 0.4162 |

*Mean organ absorbed doses and effective dose are expressed in mGy/MBq and mSv/MBq, respectively.
^{†}100 μg of A33-TCO injected 24 h before injection of ⁶⁴Cu-Tz-Bn-NOTA.
^{‡}5 μg of ⁶⁴Cu-NOTA-A33 per injection.
^{§}5 μg of ⁸⁹Zr-DFO-A33 per injection.

We claim:

1. A method of in vivo imaging, comprising a) ex vivo modification of an antibody with amine-reactive transcyclooctene to create a plurality of antibody-transcyclooctene conjugates; b) in vivo administration to a subject of the antibody-transcyclooctene conjugates wherein the subject comprises tissue reactive with said antibody; c) in vivo administration of a labeled tetrazine derivative, under conditions such that at least a portion of the administered labeled tetrazine derivative reacts with at least a portion of said plurality of conjugates to create an imaging reagent bound to said tissue reactive with said antibody; and d) imaging the tissue reactive with said antibody.

2. The method of claim 1, wherein the subject is a human and the tissue reactive with said antibody is a tumor.

3. The method of claim 2, wherein said administration of conjugate at step b) is by intravenous injection into the blood of said subject.

4. The method of claim 3, wherein prior to step c), sufficient time is provided during which a portion of said plurality of antibody conjugates binds to the tumor and at least a portion of unbound antibody conjugate clears from the blood.

5. The method of claim 1, wherein said labeled tetrazine derivative is labeled with a radiolabel.

6. The method of claim 1, wherein said antibody of step a) is a monoclonal antibody.

7. The method of claim 6, wherein said monoclonal antibody is a humanized antibody.
